(19) United States
(12) Patent Application Publication
(10) Pub. No.: US 2025/0290152 A1
(43) Pub. Date: Sep. 18, 2025

(54) GENETIC TRIO OF BRAF AND TERT MUTATIONS AND RS2853669TT IN PAPILLARY THYROID CANCER AGGRESSIVENESS

(71) Applicant: The Johns Hopkins University, Baltimore, MD (US)

(72) Inventors: Michael Mingzhao Xing, Clarksville, MD (US); Rengyun Liu, Baltimore, MD (US)

(21) Appl. No.: 18/988,566

(22) Filed: Dec. 19, 2024

Related U.S. Application Data
(60) Provisional application No. 63/612,153, filed on Dec. 19, 2023.

(51) Int. Cl. C12Q 1/6886

(52) U.S. Cl. CPC C12Q 1/6886; C12Q 2600/118; C12Q 2600/156; G01N 2800/52

(57) ABSTRACT

The present invention relates to the field of cancer. More specifically, the present invention provides methods and compositions related to certain mutations in thyroid cancer. The present inventors hypothesized that SNP rs2853669C>T, by affecting the TERT promoter activities, could differentiate the disease aggressiveness risk associated with BRAF V600E and TERT promoter mutations in PTC and therefore refine their prognostic precision. The combination of these genetic variants in BRAF and TERT genes represents a simple but effective genetic risk prognostication strategy for PTC.

### CROSS-REFERENCE TO RELATED APPLICATIONS

This application claims the benefit of U.S. Provisional Application No. 63/612,153, filed Dec. 19, 2023, which is incorporated herein by reference in its entirety.

### GOVERNMENT SUPPORT CLAUSE

This invention was made with government support under grant no. CA189224 and grant no. CA215142, awarded by the National Institutes of Health. The government has certain rights in the invention.

### FIELD OF THE INVENTION

The present invention relates to the field of cancer. More specifically, the present invention provides methods and compositions useful for the treatment of cancer characterized by TERT and BRAF mutations.

### REFERENCE TO AN ELECTRONIC SEQUENCE LISTING

The text of the computer readable sequence listing filed herewith, titled “P18157-02”, created Dec. 18, 2024, having a file size of 16,069 bytes, is hereby incorporated by reference in its entirety.

### BACKGROUND OF THE INVENTION

Papillary thyroid cancer (PTC) is common, accounting for nearly 90% of all thyroid cancers (1, 2). Clinical prognosis of PTC varies widely, with 10-15% of cases being inherently aggressive with high recurrence and mortality and the remainder of cases being generally indolent, making accurate risk assessment important to balance the benefits of treatment against the risks associated with them (3, 4). This can be assisted by molecular-based risk assessment of thyroid cancer, particularly PTC, in which genetic-based risk prognostication and precision management are becoming a reality (3, 5-8). In this regard, two prominent prognostic genetic markers, BRAF V600E and telomerase reverse transcriptase (TERT) gene promoter mutations, play a central role. BRAF V600E is the most common oncogenic mutation in PTC, which exerts oncogenicity through constitutively activating the mitogen-activated protein kinase (MAPK) pathway (9). There are two common TERT promoter mutations, 1,295,228 C>T (C228T) and 1,295,250 C>T (C250T) (10, 11). They generate consensus binding sites for the transcriptional GA binding protein (GABP) complex to bind and oncogenically activate TERT (12, 13). The BRAF and TERT mutations have been widely reported to be associated with tumor aggressiveness and poor clinical outcomes of PTC (14-24). A notable phenomenon of BRAF V600E and TERT promoter mutations is their common coexistence to form a distinct genetic duet in PTC, as initially reported in 2013 (17); this coexistence has been consistently found to be associated with tumor aggressiveness, disease recurrence and patient mortality in PTC (19, 20, 22, 24-35). This genetic duet largely distinguishes the 10-15% of patients with aggressive PTC from the majority with indolent disease (22, 28, 36). Therefore, BRAF V600E and TERT promoter mutations are currently the essential components of genetic-based risk prognostication strategies for PTC, which are increasingly recognized and advocated for precision management of PTC (3, 7, 10, 11, 37-39).

### SUMMARY OF THE INVENTION

The present inventors hypothesized that the prognostic value of BRAF V600E and TERT promoter mutations in papillary thyroid cancer (PTC) could be refined by the SNP rs2853669C>T in the TERT gene. As described herein, the genetic trio of co-existing BRAF V600E, TERT promoter mutation and TT of rs2853669 is associated with the highest tumor recurrence of PTC whereas lack of them is associated with the lowest recurrence. Thus, the introduction of SNP rs2853669 into the current prognostic use of the genetic duet of BRAF and TERT mutations may make the genetic-based risk prognostication even more precise in PTC.

In one aspect, the present invention provides methods for treating a subject having aggressive thyroid cancer. In one embodiment, the method the steps of (a) performing an assay on a sample obtained from the subject to identify a (i) a T1799A mutation in the v-raf murine sarcoma viral oncogene homolog B1 (BRAF) gene that results in a V600E amino acid change, (ii) a genotype of TT at the single nucleotide polymorphism (SNP) rs2853669; and either (iii) a 1 295 228 C>T (C228T) mutation, corresponding to −124 C>T from the translation start site in the promoter of the telomerase reverse transcriptase (TERT) gene, or (iv) a 1 295 250 C>T (C250T) mutation, corresponding to −146 C>T from the translation start site in the promoter of TERT; (b) identifying the subject as having or likely to develop aggressive thyroid cancer when the V600E mutation, genotype of TT at rs2853669 and either the C228T or C250T mutations are identified; and (c) treating the subject with one or more treatment modalities appropriate for a subject having or likely to develop aggressive thyroid cancer.

In a specific embodiment, the assay of step (a) comprises sequencing of the BRAF gene that comprises the T1799A nucleotide site and sequencing of the TERT promoter region comprising −124, −146 and −245 from the translation start site in the promoter of TERT.

In a more specific embodiment, the assay of step (a) comprises the steps of (i) extracting DNA from the biological sample; (ii) contacting the DNA with a primer that specifically hybridizes to the BRAF gene and a primer that specifically hybridizes to the TERT gene; (iii) amplifying by polymerase chain reaction (PCR) a region of the BRAF gene that comprises the T1779A nucleotide site and a region of the TERT gene that comprises −124, −146 and −245 from the translation start site in the promoter of TERT; and (iv) sequencing the amplification product to identify the presence of the V600E mutation, genotype of TT at rs2853669, and either the C228T or C250T mutation.

In specific embodiments, the treatment modality for aggressive thyroid cancer comprises thyroidectomy, hemithyroidectomy, radioactive iodine therapy, and combinations thereof. In an alternative embodiment, the treatment modality comprises administering to the subject a BRAF V600E inhibitor. In another embodiment, the treatment modality comprises administering to the subject a MEK inhibitor. In yet another embodiment, the treatment modality comprises administering to the subject a TERT inhibitor. In other embodiments, the treatment modality comprises administering to the subject a FOS inhibitor. In particular embodiments, the treatment modality comprises administering to the subject both BRAF V600E/MEK inhibitors and TERT inhibitor. In certain embodiments, the aggressive thyroid cancer is papillary thyroid cancer (PTC) or anaplastic thyroid cancer.

In another aspect, the present invention provides methods for identifying a subject as having or likely to develop aggressive thyroid cancer. In one embodiment, the method comprises the steps of (a) performing an assay on a sample obtained from the subject to identify a (i) a T1799A mutation in the BRAF gene that results in a V600E amino acid change, (ii) a genotype of TT at the SNP rs2853669; and either (iii) a 1 295 228 C>T (C228T) mutation, corresponding to −124 C>T from the translation start site in the promoter of the TERT gene, or (iv) a 1 295 250 C>T (C250T) mutation, corresponding to −146 C>T from the translation start site in the promoter of TERT; and (b) identifying the subject as having or likely to develop aggressive thyroid cancer when the V600E mutation, genotype of TT at rs2853669 and either the C228T or C250T mutations are identified.

In a specific embodiment, the assay of step (a) comprises sequencing of the BRAF gene that comprises the T1799A nucleotide site and sequencing of the TERT promoter region comprising −124, −146 and −245 from the translation start site in the promoter of TERT.

In a more specific embodiment, the assay of step (a) comprises the steps of (i) extracting DNA from the biological sample; (ii) contacting the DNA with a primer that specifically hybridizes to the BRAF gene and a primer that specifically hybridizes to the TERT gene; (iii) amplifying by polymerase chain reaction (PCR) a region of the BRAF gene that comprises the T1779A nucleotide site and a region of the TERT gene that comprises −124, −146 and −245 from the translation start site in the promoter of TERT; and (iv) sequencing the amplification product to identify the presence of the V600E mutation, genotype of TT at rs2853669, and either the C228T or C250T mutation.

In certain embodiments, the method further comprises the step of administering a treatment modality appropriate for a subject having or likely to develop aggressive thyroid cancer. In specific embodiments, the treatment modality for aggressive thyroid cancer comprises thyroidectomy, hemithyroidectomy, radioactive iodine therapy, and combinations thereof. In a further embodiment, the treatment modality comprises administering to the subject a BRAF V600E inhibitor. In yet another embodiment, the treatment modality comprises administering to the subject a TERT inhibitor. In particular embodiments, the aggressive thyroid cancer is papillary thyroid cancer (PTC) or anaplastic thyroid cancer. In another embodiment, the treatment modality comprises administering to the subject a MEK inhibitor. In other embodiments, the treatment modality comprises administering to the subject a FOS inhibitor. In certain embodiments, the treatment modality comprises administering to the subject both BRAF V600E/MEK inhibitors and TERT inhibitor.

In particular embodiments, the biological sample is from a fine needle aspiration biopsy. In specific embodiments, the amplification of the TERT gene is accomplished using one or more primers shown in SEQ ID NOS:1-7. In other embodiments, the amplification of the BRAF gene is accomplished using on or more primers shown in SEQ ID NOS:8-13. In particular embodiments, a portion of the TERT promoter that comprises rs2853669 and the sites of the C228T and C250T mutations is amplified.

The present invention demonstrates that the SNP rs2853669C>T can differentiate the disease aggressiveness risk associated with BRAF V600E and TERT promoter mutations in PTC and therefore refine their prognostic precision. The combination of these genetic variants in BRAF and TERT genes represents a simple but effective genetic risk prognostication strategy for PTC.

### BRIEF DESCRIPTION OF THE FIGURES

FIG. 1A-1B. Kaplan-Meier analysis of the synergistic associations of genetic variants with disease-free survival of patients with papillary thyroid cancer. (FIG. 1A) synergistic effects of BRAF V600E and TERT promoter mutations. (FIG. 1B) synergistic effects of BRAF V600E, TERT promoter mutations, and genotype TT of rs2853669. The curves are truncated at 20 years of follow-up.

FIG. 2. Luciferase report assay of activities of the TERT promoter with various genetic conditions. Luciferase reporter constructs containing various genetic variant combinations of the TERT promoter were transfected together with Renilla luciferase plasmid into papillary thyroid cancer cell-derived TPC1 cells for 24 hours, followed by measurement of the luciferase activities using the Dual-Luciferase Reporter Assay System. **P<0.01, ***P<0.001 from the independent t test.

FIG. 3A-3C. Kaplan-Meier analyses of the effects of rs2853669, BRAF V600E, and TERT promoter mutations on disease-free survival of patients with PTC. (FIG. 3A) Results of the analyses of rs2853669. (FIG. 3B) Results of the analyses of BRAF V600E mutation. (FIG. 3C) Results of the analyses of TERT promoter mutations. The curves are truncated at 20 years of follow-up.

FIG. 4. Kaplan-Meier analysis of the synergistic effects of genetic variants for disease-free survival of patients with conventional PTC. The curves are truncated at 20 years of follow-up.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term “about.”

An “agonist” is a type of modulator and refers to an agent that binds a target and can activate one or more functions of the target. For example, an agonist of a protein can bind the protein and activate the protein in the absence of its natural or cognate ligand.

As used herein, an “antagonist” is a type of modulator and is used interchangeably with the term “inhibitor.” In certain non-limiting embodiments, the term refers to an agent that binds a target (e.g., a protein) and can inhibit a one or more functions of the target. For example, an antagonist of an enzymatic protein can bind the protein and inhibit the enzymatic activity of the protein.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies. In specific embodiments, antibodies may be raised against TERT and used as TERT modulators. In other embodiments, antibodies may be raised against BRAF and used as BRAF modulators.

As used herein, the term “effective,” means adequate to accomplish a desired, expected, or intended result. More particularly, a “therapeutically effective amount” as provided herein refers to an amount of a TERT and/or BRAF modulator of the present invention, either alone or in combination with another therapeutic agent, necessary to provide the desired therapeutic effect, e.g., an amount that is effective to prevent, alleviate, or ameliorate symptoms of disease or prolong the survival of the subject being treated. In a specific embodiment, the term “therapeutically effective amount” as provided herein refers to an amount of a TERT and/or BRAF modulator, necessary to provide the desired therapeutic effect, e.g., an amount that is effective to prevent, alleviate, or ameliorate symptoms of disease or prolong the survival of the subject being treated. In a particular embodiment, the disease or condition is cancer. In a more specific embodiment, the cancer is thyroid cancer. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

The term “inhibitor” is a type of modulator and is used interchangeably with the term “antagonist.” The term “inhibitor” includes any type of molecule or agent that directly or indirectly inhibits the expression or activity of a target gene or protein. An inhibitor can be any type of compound, such as a small molecule, antibody or antisense compound. In certain embodiments, the target gene or protein is TERT. The term also includes agents that have activity in addition to TERT inhibitory activity. In another embodiment, the target gene or protein is BRAF. The term also includes agents that have activity in addition to BRAF inhibitory activity. Examples of BRAF inhibitors include Sorafenib (Bay 43-9006, Nexavar), Vemurafenib (PLX4032), BDC-0879, PLX-4720, Dabrafenib (Tafinlar), and LGX818. BRAF inhibitors further include, but are not limited to, the combination of Dabrafenib (Tafinlar) and trametinib (Mekinist); BRAFTOVI taken in combination with cetuximab; kinase inhibitor R05185426; and encorafenib (with MEK inhibitor, binimetinib). In still another embodiment, the target gene or protein is MEK, a protein downstream BRAF in the BRAF/MEK/MAP kinase pathway (Mitogen-activated protein kinase kinase—also known as MAP2K, MEK, MAPKK). Examples of MEK inhibitors include trametinib, selumetinib (AZD6244), trametinib, CI1040, PD0325901, RDEA119 (refametinib, BAY 869766). TERT inhibitors include, but are not limited to, NU-1 (Northwestern University) (See, e.g., claims 1-16 of U.S. Pat. No. 11,518,750); IVS-2001 (Invectys); INO-1400, INO-1401 and INO-5401 (Inovio); and 6-thio-2′-deoxyguanosine (THIO). Synthetic direct telomerase inhibitors include MST-312 [N,N′-bis(2,3-dihydroxybenzoyl)-1,2-phenylenediamine, dihydroxybenzoyl-1,3-phenylenediamine]and MST-199 (N-[2-(3,4-dihydroxyphenyl)-4-oxo-4H-chromen-3-yl]-3,4 dihydroxybenzamide). Synthetic indirect telomerase inhibitors include acridine compounds (e.g., geldanamycin, GA alkyn and bis-amido chloroacridine). Other TERT inhibitors include eight platinum complexes with substituted 3-(2′-benzimidazolyl) coumarins; N-substituted-dihydropyrazole derivatives; Silibinin, a polyphenolic flavonoid; ethenesulfonyl fluoride derivatives, including certain 2-(hetero) arylethenesulfonyl fluoride and 1,3-dienylsulfonyl fluoride derivatives; Imidazole-4-one derivatives and imetelstat (GRN163L). Finally, small interfering RNAs (siRNAs) are a novel strategy for silencing the hTERT expression. For example, Ghareghomi et al. indicated that siRNAs in combination with magnetic nanoparticles may be an effective treatment for ovarian cancer. See Ghareghomi et al., 277 LIFE SCI. 1196212021 (2021) (“hTERT-molecular targeted therapy of ovarian cancer cells via folate-functionalized PLGA nanoparticles co-loaded with MNPs/siRNA/wortmannin”). In still another embodiment, the combination use of BRAF and TERT inhibitors targeting both genes or proteins is more effective. In still another embodiment, the treatment targets simultaneously TERT and BRAF/MEK using their corresponding inhibitors.

As used herein, the term “modulate” indicates the ability to control or influence directly or indirectly, and by way of non-limiting examples, can alternatively mean inhibit or stimulate, agonize or antagonize, hinder or promote, and strengthen or weaken. Thus, the term “TERT modulator” refers to an agent that modulates the expressions and/or activity of TERT. The term “BRAF modulator” refers to an agent that modulates the expressions and/or activity of BRAF. Modulators may be organic or inorganic, small to large molecular weight individual compounds, mixtures and combinatorial libraries of inhibitors, agonists, antagonists, and biopolymers such as peptides, nucleic acids, or oligonucleotides. A modulator may be a natural product or a naturally-occurring small molecule organic compound. In particular, a modulator may be a carbohydrate; monosaccharide; oligosaccharide; polysaccharide; amino acid; peptide; oligopeptide; polypeptide; protein; receptor; nucleic acid; nucleoside; nucleotide; oligonucleotide; polynucleotide including DNA and DNA fragments, RNA and RNA fragments and the like; lipid; retinoid; steroid; glycopeptides; glycoprotein; proteoglycan and the like; and synthetic analogues or derivatives thereof, including peptidomimetics, small molecule organic compounds and the like, and mixtures thereof. A modulator identified according to the invention is preferably useful in the treatment of a disease disclosed herein.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

Optional” or “optionally” means that the subsequently described event or circumstance can or cannot occur, and that the description includes instances where the event or circumstance occurs and instances where it does not.

The terms “patient,” “individual,” or “subject” are used interchangeably herein, and refer to a mammal, particularly, a human. The patient may have a mild, intermediate or severe disease or condition. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates. In particular, the term also includes mammals diagnosed with a BRAF and/or TERT mediated disease, disorder or condition. By “normal subject” is meant an individual who does not have cancer as well as an individual who has increased susceptibility for developing a cancer.

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules. In addition, as used herein, the term “polypeptide” refers to amino acids joined to each other by peptide bonds or modified peptide bonds, e.g., peptide isosteres, etc., and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983).

By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for TERT and/or BRAF nucleic acids (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the TERT or BRAF nucleic acid to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

The terms “sample,” “patient sample,” “biological sample,” and the like, encompass a variety of sample types obtained from a patient, individual, or subject and can be used in a diagnostic or monitoring assay. The patient sample may be obtained from a healthy subject or a patient having symptoms associated with prostate cancer. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiments, a sample comprises blood. In other embodiments, a sample comprises serum. In a specific embodiment, a sample comprises plasma. In yet another embodiment, a semen sample is used. In a further embodiment, a stool sample is used. In particular embodiments, TERT promoter and/or BRAF mutations described here can be tested on tumor tissues, including surgical tissues, needle biopsy tissues (e.g., thyroid nodule needle biopsy specimens), body fluids (e.g., needle biopsy washings, cerebral spinal fluids, urine, etc.) for the diagnosis, prognosis and treatment guidance and treatments of cancer, such as thyroid cancer and other cancers that harbor the BRAF and TERT mutations described herein, as well as the SNP rs2853669T>C. In certain embodiments, the sample is from a fine needle aspiration biopsy.

In certain embodiments, a sample comprises urine. Indeed, TERT mutations can be detected in urine as molecular markers for the diagnosis, prognostication and treatment of bladder cancer. See Hurst et al., 65 European Urology 367-69 (2014) (“Comprehensive Mutation Analysis of the TERT Promoter in Bladder Cancer and Detection of Mutations in Voided Urine”); and Rochakonda et al., 110(43) Proc. Natl. Acad. Sci. USA 17426-17431 (October 2013) (“TERT Promoter Mutations in Bladder Cancer Affect Patient Survival and Disease Recurrence Through Modification by a Common Polymorphism”).

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

The terms “specifically binds to,” “specific for,” and related grammatical variants refer to that binding which occurs between such paired species as antibody/antigen, enzyme/substrate, receptor/agonist, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

By “specifically hybridizes” is meant that a probe, primer, or oligonucleotide recognizes and physically interacts (that is, base-pairs) with a substantially complementary nucleic acid (for example, a TERT nucleic acid) under high stringency conditions, and does not substantially base pair with other nucleic acids.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease. In a specific embodiment, the disease or condition is cancer. In particular embodiments, the cancer is thyroid cancer. In further embodiments, the cancer includes bladder and glioblastoma. Treatment can include administration of a BRAF inhibitor, MEK inhibitor, TERT inhibitor, FOS inhibitor and the like.

The terms “TERT-related disease, disorder or condition” or “TERT-mediated disease, disorder or condition,” and the like mean diseases, disorders or conditions associated with aberrant TERT activity. In a specific embodiment, the disease or condition is cancer. In a more specific embodiment, the cancer is thyroid cancer. In general, the term refers to any abnormal state that involves TERT activity. The abnormal state can be due, for example, to a genetic defect.

The terms “BRAF-related disease, disorder or condition” or “BRAF-mediated disease, disorder or condition,” and the like mean diseases, disorders or conditions associated with aberrant BRAF activity. In a specific embodiment, the disease or condition is cancer. In a more specific embodiment, the cancer is thyroid cancer. In general, the term refers to any abnormal state that involves BRAF activity. The abnormal state can be due, for example, to a genetic defect.

### II. BRAF V600E and TERT Promoter Mutations as Biomarkers

The present inventors have discovered that BRAF V600E and certain mutations in the promoter region of TERT provide a unique genetic background that predict and identify the most aggressive cases of human thyroid cancers. Thyroid cancer can include follicular thyroid cancer (FTC), papillary thyroid cancer (PTC), conventional PTC, follicular variant PTC (FVPTC), tall-cell PTC (TCPTC). In particular embodiments, the mutations are used to predict and identify and/or treat the most aggressive type of PTC.

Thus, in certain embodiments, the BRAF and TERT promoter mutations can thus be used to identify individuals having or at risk of developing cancer, in particular, aggressive cancer. In further embodiments, the BRAF and TERT promoter mutations can be used to identify individuals at risk for having or developing aggressive thyroid cancer such as TCPTC, PDTC, ATC and PTC. In certain embodiments, the aggressive thyroid cancer is PTC. The mutations can be identified in subjects who have or have not been diagnosed with cancer. In other embodiments, methods and compositions described herein can be used to examine BRAF and TERT promoter mutations other cancers including melanoma, colon cancer, brain tumor, leukemia (particularly hairy cell leukemia), lungs cancer, ovarian cancer, uterine cancer, cervical cancer, nasopharyngeal cancer, pancreatic cancer, and papillary craniopharyngiomas.

In certain embodiments, DNA can be isolated from a biological sample taken from a subject. DNA can be extracted and purified from biological samples using any suitable technique. A number of techniques for DNA extraction and/or purification are known in the art, and several are commercially available (e.g., ChargeSwitch®, MELT™ total nucleic acid isolation system, MagMAX™ FFPE total nucleic acid isolation kit, MagMAX™ total nucleic acid isolation kit, QIAamp DNA kit, Omni-Pure™ genomic DNA purification system, WaterMaster™ DNA purification kit). Reagents such as DNAzoI® and TR1 Reagent@can also be used to extract and/or purify DNA. DNA can be further purified using Proteinase K and/or RNAse.

In specific embodiments, primer/probes can be used to amplify a region of the TERT gene that comprises the promoter. More specifically, primers/probes are capable of amplifying the promoter region at 1 295 228 C>T and/or 1 295 250 C>T (termed C228T and C250T respectively), corresponding to −124 C>T and −146 C>T from the translation start site in the promoter of the telomerase reverse transcriptase (TERT) gene. In one embodiment, a primer comprises the nucleic acid sequence shown in SEQ ID NO:1. In another embodiment, a primer comprises the nucleic acid sequence shown in SEQ ID NO:2. A primer set can comprise the nucleic acid sequences shown in SEQ ID NO:1 and SEQ ID NO:2.

Thus, in a specific embodiment, the TERT primers comprise TERT sense: AGTGGATTCGCGGGCACAGA (SEQ ID NO:1) and TERT antisense: CAGCGCTGCCTGAAACTC (SEQ ID NO:2. In another embodiment, primers for TERTp can comprise: forward, 5′-AGTGGATTCGCGGGCACAGA-3′ (SEQ ID NO:1), and reverse, 5′-AGCACCTCGCGGTAGTGG-3′ (SEQ ID NO:3), which amplifies a 346 bp fragment. In yet another embodiment, the sequences of the forward and reverse primers comprise 5′-CACCCGTCCTGCCCCTTCACCTT-3′ (SEQ ID NO:4) and 5′-GGCTTCCCACGTGCGCAGCAGGA-3′ (SEQ ID NO:5), respectively. The forward and reverse primers can also comprise 5′-CCAAGTTCCTGCACTGGCTGA-3′ (SEQ ID NO:6) and 5′-TTCCCGATGCTGCCTGAC-3′ (SEQ ID NO:7), respectively. The region of the TERT promoter comprising the locus of the relevant mutations can be amplified using any one or more of SEQ ID NOS:1-7.

In particular embodiments, a single set of primers is used to amplify a region of the TERT promoter that includes the relevant mutations: C228T, C250T and the SNP rs2853669T>C. The amplified region of the TERT promoter would include −245, −124 and −146. One example of such a primer set comprises SEQ ID NO:1 and SEQ ID NO:3, which produces a 343 bp fragment comprising the relevant loci.

In certain embodiments, primer/probes can be used to amplify a region of the BRAF gene comprising the site for the T1799A (V600E) mutation. In one embodiment, a primer comprises the nucleic acid sequence shown in SEQ ID NO:8. In another embodiment, a primer comprises the nucleic acid sequence shown in SEQ ID NO:9. A primer set can comprise the nucleic acid sequences shown in SEQ ID NO:8 and SEQ ID NO:9.

Thus, in a specific embodiment, the BRAF primers comprise BRAF sense: TCATAATGCTTGCTCTGATAGGA (SEQ ID NO:8); and BRAF antisense: GGCCAAAAATTTAATCAGTGGA (SEQ ID NO:9). In another embodiment, the DNA sequence around the BRAF V600 region can be amplified from genomic DNA using primer pair with following sequences 5′-TGTAAAACGACGGCCAGTCTGTTTTCCTTTACTTACTACACCTCAGAT-3′ (SEQ ID NO:10) and 5′-CAACTGTTCAAACTIGATGGG-3 (SEQ ID NO:11). An M13 forward primer adaptor sequence (labeled with underline) can be incorporated into the forward primer to facilitate sequencing.

In yet another embodiment, a set of PCR primers 5′-AACTCTTCATAATGCTTGCTCTGA-3′ (SEQ ID NO:12) and 5′-CAGACAACTGTTCAAACTGATGGGACC-3′ (SEQ ID NO:13) can be used to amplify a region of human gDNA encompassing the BRAF V600 locus. The amplified product is 180 base pairs (bp). Two sequencing primers can be designed to recognize the respective single nucleotide at their 3′ end independently, one for BRAF mutant V600E and the other for BRAF wild type V600. These two sequencing primers differ in two respects: the nucleotide at their 3′ end and the respective molecular weight. Allele specific nucleotide at their 3′ end determines their respective specificity of the two sequence primers. For example, BRAF V600E mutant harbors a deoxythymidine and V600 wild-type carries a deoxyadenosine. The different molecular weights of the allele-specific sequencing primer separates the truncated molecules generated from the mutant sequencing primer from the wild type sequencing primer. Allele-specific sequencing primers comprise (mutant) V600E-SP: 5′-AATAGGTGATTTTGGTCTAGCTACAGT-3′ (SEQ ID NO:14) and (wild type) V600-SP: 5′-weighted-AATAGGTGATTTTGGTCTAGCTACAGA-3′ (SEQ ID NO:15). The region of BRAF comprising the locus of the relevant mutation can be amplified using any one or more of SEQ ID NOS:8-13.

In particular embodiments, a primer is contacted with isolated DNA from the subject under conditions such that the primer specifically hybridizes with the TERT or BRAF genes. The primer and DNA thus form a primer:DNA complex. In certain embodiments, the hybridization conditions are such that the formation of the primer:DNA complex is the detection step itself, i.e., the complex forms only if the mutation (TERT C228T, TERT C250T and/or BRAF T1799A (V600E)) is present. In other embodiments, the primer:DNA complex is amplified using polymerase chain reaction, the presence (or not) of the mutation is detected. In certain embodiments, the mutations are detected by sequencing.

As described herein, in certain embodiments, the primers can used to support DNA amplification reactions. Typically the primers will be capable of being extended in a sequence specific manner. Extension of a primer in a sequence specific manner includes any methods wherein the sequence or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the polynucleotide sequences disclosed herein or region of the polynucleotide sequences disclosed herein or they hybridize with the complement of the polynucleotide sequences disclosed herein or complement of a region of the polynucleotide sequences disclosed herein.

The size of the primers or probes for interaction with the polynucleotide sequences disclosed herein in certain embodiments can be any size that supports the desired enzymatic manipulation of the primer, such as DNA amplification or the simple hybridization of the probe or primer. A typical primer or probe would be at least 6, 7, 8, 9, 10, 20, 30, 40, 50, 60, 70, 80, 90, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long or any length in-between.

Primers specific for amplification of the TERT gene can be designed to produce amplification products that comprise the TERT C228T and/or TERT C250T locus. Similarly, primers for amplification of the BRAF gene can be designed to produce amplification products that comprise the T1799A (V600E) locus. Such amplification products can be of any suitable length including, but not limited to, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, and 300 or more nucleotides.

The probes or primers of the present invention can be prepared by conventional techniques well-known to those skilled in the art. For example, the probes can be prepared using solid-phase synthesis using commercially available equipment. Modified oligonucleotides can also be readily prepared by similar methods. The probes can also be synthesized directly on a solid support according to methods standard in the art. This method of synthesizing polynucleotides is particularly useful when the polynucleotide probes are part of a nucleic acid array.

### III. Genotyping TERT Polymorphism rs2863669

In particular embodiments, a TERT nucleic acid is amplified by PCR to determine the genotype of the isoform, e.g., TT/TC/CC genotype of rs2853669. The amplified nucleic acid may be analyzed using a variety of methods for detecting variant alleles to determine the genotype. The presence or absence of a polymorphism in the TERT promoter may be evaluated using various techniques. For example, the TERT promoter is amplified by PCR and sequenced to determine the presence or absence of a single nucleotide polymorphism (SNP). In certain embodiments, real-time PCR may be used to detect a single nucleotide polymorphism of the amplified products. In other embodiments, a polymorphism in the amplified products may be detected using a technique including hybridization with a probe specific for a single nucleotide polymorphism, restriction endonuclease digestion, primer extension, microarray or gene chip analysis, mass spectrometry, or a DNAse protection assay.

Various PCR testing platforms that may be used with the present invention include: 5′ nuclease (TaqMan® probes), molecular beacons, and FRET hybridization probes. These detection methods rely on the transfer of light energy between two adjacent dye molecules, a process referred to as fluorescence resonance energy transfer.

In certain embodiments, a 5′ nuclease probe may be used to detect a polymorphism of the present invention. 5′ nuclease probes are often referred to by the proprietary name, TaqMan® probes. A TaqMan® probe is a short oligonucleotide (DNA) that contains a 5′ fluorescent dye and 3′ quenching dye. To generate a light signal (i.e., remove the effects of the quenching dye on the fluorescent dye), two events must occur. First, the probe must bind to a complementary strand of DNA, e.g., at about 60° C. Second, at this temperature, Taq polymerase, which is commonly used for PCR, must cleave the 5′ end of the TaqMan® probe (5′ nuclease activity), separating the fluorescent dye from the quenching dye.

In order to differentiate a single nucleotide polymorphism from a wild-type sequence in the DNA from a subject, a second probe with complementary nucleotide(s) to the polymorphism and a fluorescent dye with a different emission spectrum are typically utilized. Thus, these probes can be used to detect a specific, predefined polymorphism under the probe in the PCR amplification product. Two reaction vessels are typically used, one with a complementary probe to detect wild-type target DNA and another for detection of a specific nucleic acid sequence of a mutant strain. Because TaqMan® probes typically require temperatures of about 60° C. for efficient 5′ nuclease activity, the PCR may be cycled between about 90-95° C. and about 60° C. for amplification. In addition, the cleaved (free) fluorescent dye can accumulate after each PCR temperature cycle; thus, the dye can be measured at any time during the PCR cycling, including the hybridization step. In contrast, molecular beacons and FRET hybridization probes typically involve the measurement of fluorescence during the hybridization step.

Genotyping for the polymorphism in the TERT promoter may be evaluated using the following (5′ endonuclease probe) real-time PCR technique. Genotyping assays can be performed in duplicate and analyzed on a Bio-Rad iCycler Iq® Multicolor Real-time detection system (Bio-Rad Laboratories, Hercules, Calif.). Real-time polymerase chain reaction (PCR) allelic discrimination assays to detect the presence or absence of specific single nucleotide polymorphisms in a TERT promoter may utilize fluorogenic TaqMan® Probes.

Real-time PCR amplifications may be carried out in a 10 μl reaction mix containing 5 ng genomic DNA, 900 Nm of each primer, 200 Nm of each probe and 5 μl of 2× TaqMan® Universal PCR Master Mix (contains PCR buffer, passive reference dye ROX, deoxynucleotides, uridine, uracil-N-glycosylase and AmpliTaq Gold DNA polymerase; Perkin-Elmer, Applied Biosystems, Foster City, Calif.). Cycle parameters may be: 95° C. for 10 min, followed by 50 cycles of 92° C. for 15 sec and 60° C. for 1 min. Real-time fluorescence detection can be performed during the 60° C. annealing/extension step of each cycle. The IQ software may be used to plot and automatically call genotypes based on a two parameter plot using fluorescence intensities of FAM and VIC at 49 cycles.

### C. Molecular Beacons

Molecular beacons are another real-time PCR approach which may be used to identify the presence or absence of a polymorphism of the present invention. Molecular beacons are oligonucleotide probes that are labeled with a fluorescent dye (typically on the 5′ end) and a quencher dye (typically on the 3′ end). A region at each end of the molecular beacon probe is designed to be complementary to itself, so at low temperatures the ends anneal, creating a hairpin structure. This hairpin structure positions the two dyes in close proximity, quenching the fluorescence from the reporter dye. The central region of the probe is designed to be complementary to a region of a PCR amplification product. At higher temperatures, both the PCR amplification product and probe are single stranded. As the temperature of the PCR is lowered, the central region of the molecular beacon probe may bind to the PCR product and force the separation of the fluorescent reporter dye from the quenching dye. Without the quencher dye in close proximity, a light signal from the reporter dye can be detected. If no PCR amplification product is available for binding, the probe can re-anneal to itself, bringing the reporter dye and quencher dye into close proximity, thus preventing fluorescent signal.

Two or more molecular beacon probes with different reporter dyes may be used for detecting single nucleotide polymorphisms. For example, a first molecular beacon designed with a first reporter dye may be used to indicate the presence of a SNP and a second molecular beacon designed with a second reporter dye may be used to indicate the presence of the corresponding wild-type sequence; in this way, different signals from the first and/or second reporter dyes may be used to determine if a subject is heterozygous for a SNP, homozygous for a SNP, or homozygous wild-type at the corresponding DNA region. By selection of appropriate PCR temperatures and/or extension of the probe length, a molecular beacons may bind to a target PCR product when a nucleotide polymorphism is present but at a slight cost of reduced specificity. Molecular beacons advantageously do not require thermocycling, so temperature optimization of the PCR is simplified.

### D. FRET Hybridization Probes

FRET hybridization probes, also referred to as LightCycler® probes, may also be used to detect a polymorphism of the present invention. FRET hybridization probes typically comprise two DNA probes designed to anneal next to each other in a head-to-tail configuration on the PCR product. Typically, the upstream probe has a fluorescent dye on the 3′ end and the downstream probe has an acceptor dye on the 5′ end. If both probes anneal to the target PCR product, fluorescence from the 3′ dye can be absorbed by the adjacent acceptor dye on the 5′ end of the second probe. As a result, the second dye is excited and can emit light at a third wavelength, which may be detected. If the two dyes do not come into close proximity in the absence of sufficient complimentary DNA, then FRET does not occur between the two dyes. The 3′ end of the second (downstream) probe may be phosphorylated to prevent it from being used as a primer by Taq during PCR amplification. The two probes may encompass a region of 40 to 50 DNA base pairs.

FRET hybridization probe technology permits melting curve analysis of the amplification product. If the temperature is slowly raised, probes annealing to the target PCR product will be reduced and the FRET signal will be lost. The temperature at which half the FRET signal is lost is referred to as the melting temperature of the probe system. A single nucleotide polymorphism in the target DNA under a hybridization FRET probe will still generate a signal, but the melting curve will display a lower Tm. The lowered Tm can indicate the presence of a specific polymorphism. The target PCR product is detected and the altered Tm informs the user there is a difference in the sequence being detected. Like molecular beacons, there is not a specific thermocycling temperature requirement for FRET hybridization probes. Like molecular beacons, FRET hybridization probes have the advantage of being recycled or conserved during PCR temperature cycling, and a fluorescent signal does not accumulate as PCR product accumulates after each PCR cycle.

### E. Primer Extension

Primer extension is another technique which may be used according to the present invention. A primer and no more than three NTPs may be combined with a polymerase and the target sequence, which serves as a template for amplification. By using less than all four NTPs, it is possible to omit one or more of the polymorphic nucleotides needed for incorporation at the polymorphic site. It is important for the practice of the present invention that the amplification be designed such that the omitted nucleotide(s) is(are) not required between the 3′ end of the primer and the target polymorphism. The primer is then extended by a nucleic acid polymerase, in a preferred embodiment by Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present.

### F. RFLP

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even strains) from one another.

Restriction endonucleases in turn are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in SNP analysis requires that the SNP affect cleavage of at least one restriction enzyme site.

### G. Mass Spectrometry

Mass spectrometry may also be used to detect a polymorphism of the present invention. By exploiting the intrinsic properties of mass and charge, mass spectrometry (MS) can resolve and confidently identify a wide variety of complex compounds. Traditional quantitative MS has used electrospray ionization (ESI) followed by tandem MS (MS/MS) while other methods utilize matrix assisted laser desorption/ionization (MALDI) followed by time of flight (TOF) MS. Methods of mass spectroscopy that may be used with the present invention include: ESI, ESI tandem mass spectroscopy (ESI/MS/MS), Secondary ion mass spectroscopy (SIMS), Laser desorption mass spectroscopy (LD-MS), Laser Desorption Laser Photoionization Mass Spectroscopy (LDLPMS), and MALDI-TOF-MS.

### H. Sequencing

Nucleic acids may be sequenced using sequencing methods such as next-generation sequencing, high-throughput sequencing, massively parallel sequencing, sequencing-by-synthesis, paired-end sequencing, single-molecule sequencing, nanopore sequencing, pyrosequencing, semiconductor sequencing, sequencing-by-ligation, sequencing-by-hybridization, RNA-Seq, Digital Gene Expression, Single Molecule Sequencing by Synthesis (SMSS), Clonal Single Molecule Array (Solexa), shotgun sequencing, Maxim-Gilbert sequencing, primer walking, and Sanger sequencing.

Sequencing methods may comprise targeted sequencing, whole-genome sequencing (WGS), lowpass sequencing, bisulfite sequencing, whole-genome bisulfite sequencing (WGBS), or a combination thereof. Sequencing methods may include preparation of suitable libraries. Sequencing methods may include amplification of nucleic acids (e.g., by targeted or universal amplification, such as PCR).

Sequencing reads can be obtained from various sources including, for example, whole genome sequencing, whole exome-sequencing, targeted sequencing, next-generation sequencing, pyrosequencing, sequencing-by-synthesis, ion semiconductor sequencing, tag-based next generation sequencing semiconductor sequencing, single-molecule sequencing, nanopore sequencing, sequencing-by-ligation, sequencing-by-hybridization, Digital Gene Expression (DGE), massively parallel sequencing, Clonal Single Molecule Array (Solexa/illumina), sequencing using PacBio, and Sequencing by Oligonucleotide Ligation and Detection (SOLiD).

In some embodiments, sequencing comprises modification of a nucleic acid molecule or fragment thereof, for example, by ligating a barcode, a unique molecular identifier (UMI), or another tag to the nucleic acid molecule or fragment thereof. Ligating a barcode, UMI, or tag to one end of a nucleic acid molecule or fragment thereof may facilitate analysis of the nucleic acid molecule or fragment thereof following sequencing. In some embodiments, a barcode is a unique barcode (i.e., a UMI). In specific embodiments, a barcode is non-unique, and barcode sequences can be used in connection with endogenous sequence information such as the start and stop sequences of a target nucleic acid (e.g., the target nucleic acid is flanked by the barcode and the barcode sequences, in connection with the sequences at the beginning and end of the target nucleic acid, creates a uniquely tagged molecule).

Sequencing reads may be processed using methods such as de-multiplexing, de-deduplication (e.g., using unique molecular identifiers, UMIs), adapter-trimming, quality filtering, GC correction, amplification bias correction, correction of batch effects, depth normalization, removal of sex chromosomes, and removal of poor-quality genomic bins.)

In various embodiments, sequencing reads may be aligned to a reference nucleic acid sequence. In one example, the reference nucleic acid sequence is a human reference genome. As examples, the human reference genome can be hg19, hg38, GrCI-138, GrCH-37, NA12878, or GM12878.

### IV. Treatment Methods

In certain embodiments, the method comprises administering a TERT inhibitor. In a specific embodiment, the TERT modulator is the antagonist BIBR1532 (2-[(E)-3-naphthen-2-yl but-2-enoylamino]benzoic acid). See Ward & Autexier, Mol. Pharmacol. 68:779-786, 2005; also J. Biol. Chem. 277(18):15566-72, 2002). TERT modulator antagonists can also include TMPyP4 (tetra-(N-methyl-4-pyridyl)porphyrin); telomerase inhibitor IX (MST312); MnTMPyp pentachloride; B3PPA; P3-Rubromycin: Trichostatin A; Costunolide; Doxorubicin; Suramin Sodum; (−)-Epigallocatchin Gallate (and other catechins); triethylene tetraamine; geldanamycin; 17-(allylamino)-17-demethoxygeldanamycin. In another enbodiment, a TERT inhibitor comprises azidothymidine (AZT).

In a further embodiment, the method comprises administering a BRAF inhibitor. Examples of BRAF inhibitors include Sorafenib (Bay 43-9006, Nexavar), Vemurafenib (PLX4032), BDC-0879, PLX-4720, Dabrafenib (Tafinlar), and Encorafenib (LGX818), RAF265 (CHIR-265) AZ628 and derivatives of the foregoing.

In yet another embodiment, the method can comprise administering a MEK inhibitor. Examples of MEK inhibitors include trametinib (GSK1120212), selumetinib (AZD6244), PD184352 (CI1040), PD0325901, RDEA119 (refametinib, BAY 869766), cobimetinib (GDC-0973, RF7420), binimetinib (MEK162, ARRY-162, ARRY-438162), Pimasertib (AS-703026), TAK-733, BI-847325, GDC-0623, PD98059, and derivatives of the foregoing.

Furthermore, the patient having the trio of mutations can also be treating with a FOS inhibitor. In particular embodiments, the Fos inhibitor can be an inhibitor of the Fos gene and/or the Fos protein.

In specific embodiments, inhibitors of Fos include, but are not limited to, curcumin, difluorinated curcumin (DFC); 3-(5-(4-(cyclopentyloxy)-2-hydroxybenzoyl)-2-((3-oxo-2,3-dihydrobenzo[d]isoxazol-6-yl)methoxy)phenyl)propanoic acid or salt thereof (T5224) (see U.S. Pat. No. 8,093,289); nordihydroguaiaretic acid (NDGA); dihydroguaiaretic acid (DHGA); and 3-[5-[4-(cyclopentyloxy)-2-hydroxybenzoyl]-2-[(3-hydroxy-1,2-benzisoxazol-6-yl)methoxy]phenyl]propionic acid or salt thereof (SRi1302, Tocris Biosciences).

In further embodiments, the FOS inhibitor comprises gefitinib or erlotinib (Jimeno et al., 66(4) CANCER RES. 2385-90 (2006)).

In particular embodiments, the FOS inhibitor is a benzophenone derivative including, but not limited to, T-5224. See U.S. Pat. No. 7,772,285, which is fully incorporated herein by reference. Examples of other benzophenone derivatives include: 3-{5-[4-(cyclopentyloxy)-2-hydroxybenzoyl]-2-[(3-hydroxyl-1,2-benzisoxazo- 1-6-yl)methoxy}phenyl}propanoic acid; 2-(4-morpholinyl)ethyl 3-{5-[4-(cyclopentyloxy)-2-hydroxybenzoyl]-2-[(3-hydroxy-1,2-benzisoxazol-6-yl)methoxy]phenyl}propanoate; 4-({2-(2-carboxyethyl)-4-[4-(cyclopentyloxy)-2-hydroxybenzoyl]phenoxy}met-hyl)benzoic acid; and 3-(5-[4-(cyclopentyloxy)-2-hydroxybenzoyl]-2-{[4-(3-hydroxy-5-isoxazolyl)-benzyl]oxy}phenyl)propanoic acid.

Further FOS inhibitors include, but are not limited to, dihydromyricetin (ampelopsin, (2R,3R)-3,5,7-trihydroxy-2-(3,4,5-trihydroxyphenyl)-2,3-dihydrochromen-4-one); 3,9-bis((ethylthio)methyl)-K-252a; anthra[1,9-cd]pyrazole-6-(2H)-one (SP600125, an anthrapyrazolone ATP-competitive inhibitor); anthraquinone derivatives; (R)-4-(4-methylpentanoyl)-8-(4-methylpentylidene)-1-thia-4-azaspiro[4.5]d-ecane-3-carboxylic acid; (R)-8-(3-methylbutylidene)-4-(5-methylhexanoyl)-1-thia-4-azaspiro[4.5]dec-ane-3-carboxylic acid; and 3-[2-isobutoxy-5-(4-isobutoxybenzoyl)phenyl)propionic acid (Tsuchida et al., 49 J. MED. CHEM. 80-91 (2006)

In other embodiments, the FOS inhibitor comprises a derivative of retinoic acid including, but not limited to, SR11302 ((2E,4E,6Z,8E)-3-methyl-7-(4-me-hlylphenyl)-9-(2,6,6-trimethylcyclohexen-1-yl)-2,4,6,8-nonatetraenoic acid); SR11238 (2-(3,4-dihydro-4,4-dimethyl-2H-1-benzopyran-6-yl)-2-(4-carboxyphenyl)-1,3-dithiane); SR11327 ((E)-4-(2-(5,6,7,8tetrahydro-5,5,8,8tetramethyl-2-naphthalenyl)-3-phenyIpropenyl)benzoic acid); SR11220 (methyl (Z)-4-(1-acetoxy-2-(5,6,7,8-tetrahydro-5,5,8,8-tetramethyl-2-naphthalenyl)ethenyl)benzoate) and SRi1228 (5-((5,6,7,8-tetrahydro-5,5,8,8-tetramethyl-2-naphthalenyl)carbonyl)-2-naphthalenecarboxylic acid). See Fanjul et al., 372 NATURE 107-11(1994).

### V. Detection Kits

The present invention therefore also provides predictive, diagnostic, and prognostic kits comprising degenerate primers to amplify a target nucleic acid in the TERT and/or BRAF gene and instructions comprising amplification protocol and analysis of the results. The kit can comprise components for performing a PCR amplification of at least one gene comprising TERT and/or BRAF. In one embodiment, the kit comprises primers for producing amplification products that comprise the TERT C228T and/or TERT C250T locus. Such primers can include, but are not limited to, one or more of SEQ ID NOS:1-7. In specific embodiments, a primer set amplifies the C228T, C250T and rs2853669 loci. Similarly, primers for amplification of the BRAF gene can be designed to produce amplification products that comprise the T1799A (V600E) locus. In a specific embodiment, the primers comprise one or more of SEQ ID NOS:8-13. A primer set for sequencing may comprise one or more of SEQ ID NOS:14-15. In another embodiment, the kit can also comprise a biological collection/storage container. In specific embodiments, the kit comprises positive control DNA, negative control, and/or a master mix for performing PCR amplifications. In another embodiment, the kit comprises components for sequencing the amplified products. In a specific embodiment, the kit comprises a mix for forward/reverse sequencing of amplified PCR products. In certain embodiments, a separate PCR kit and a separate sequencing kit is provided. Alternatively, a kit can comprise components for both PCR amplification and sequencing. The kit can also comprise instructions for carrying out the amplification and/or sequencing protocols.

In more specific embodiments, the kit may alternatively also comprise buffers, enzymes, and containers for performing the amplification and analysis of the amplification products. The kit may also be a component of a screening, diagnostic or prognostic kit comprising other tools such as DNA microarrays. In some embodiments, the kit also provides one or more control templates, such as nucleic acids isolated from normal tissue sample, and/or a series of samples representing different variances in the TERT and/or BRAF gene.

In one embodiment, the kit provides at least one primer capable of amplifying a region of the TERT gene. The kit also comprises at least one primer capable of amplifying a region of the BRAF gene. The kit may comprise additional primers for the analysis of expression of several gene variances in a biological sample in one reaction or several parallel reactions. Primers in the kits may be labeled, for example fluorescently labeled, to facilitate detection of the amplification products and consequent analysis of the nucleic acid variances.

In one embodiment, more than one mutation/variance can be detected in one analysis. A combination kit will therefore comprise of primers capable of amplifying different segments of the TERT gene. The kit may also comprise primers capable of amplifying segments of another gene(s) including BRAF. The primers may be differentially labeled, for example, using different fluorescent labels, so as to differentiate between the variances. The primers contained within the kit may include primers selected from complementary sequences to the coding sequence of TERT or BRAF.

In certain embodiments, a patient can be diagnosed or identified by adding a biological sample (e.g., blood, serum, urine, etc.) obtained from the patient to the kit and detecting the TERT promoter mutations(s), for example, by a method which comprises the steps of: (i) collecting blood or blood serum from the patient; (ii) separating DNA from the patient's blood; (iii) adding the DNA from patient to a diagnostic kit; and, (iv) detecting (or not) the BRAF and TERT promoter mutation(s). In this exemplary method, primers are brought into contact with the patient's DNA. The formation of the primer:DNA complex can, for example, be PCR amplified and, in some embodiments, sequenced to detect (or not) the BRAF and TERT promoter mutation. In other kit and diagnostic embodiments, blood or blood serum need not be collected from the patient (i.e., it is already collected). Moreover, in other embodiments, the sample may comprise a tissue sample, urine or a clinical sample.

Without further elaboration, it is believed that one skilled in the art, using the preceding description, can utilize the present invention to the fullest extent. The following examples are illustrative only, and not limiting of the remainder of the disclosure in any way whatsoever.

### Examples

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Background: BRAF V600E and TERT promoter mutations are core components in current genetic-based risk assessment for precision management of papillary thyroid cancer (PTC). It remains unknown whether this could be refined to even better precision by a widely recognized prognostic single nucleotide polymorphism (SNP), rs2853669T>C, in the TERT promoter.

Methods: Genetic status of mutations and SNP were examined by sequencing genomic DNA from PTC in 608 patients (427 women and 181 men) aged 47 years (IQR 37-57), with a median follow-up time of 75 months (IQR 36 to 123), and their relationship with clinical outcomes was analyzed. Luciferase reporter assay was performed to examine TERT promoter activities.

Results: TERT promoter mutations showed a strong association with PTC recurrence in the presence of genotype TT of rs2853669 (adjusted HR=2.12, 95% CI 1.10-4.12) but not TC/CC (adjusted HR=1.17, 95% CI 0.56-2.41). TERT and BRAF mutations commonly coexisted and synergistically promoted PTC recurrence. With this genetic duet, TT of rs2853669 showed a robustly higher disease recurrence compared with TC/CC (adjusted HR=14.26, 95% CI 2.86-71.25). Patients with the genetic trio of BRAF V600E, TERT mutation and TT of rs2853669 had a recurrence of 76.5% versus recurrence of 8.4% with neither mutation and with TC/CC (HR=13.48, 95% CI 6.44-28.21). T allele of rs2853669 strongly increased TERT promoter, particularly the mutant promoter.

Conclusions: SNP rs2853669C>T dramatically refines the prognostic power of BRAF V600E and TERT promoter mutations to a higher precision, suggesting the need for including this SNP in the current genetic-based risk prognostication of PTC.

### Materials and Methods

Patient and clinicopathological data. A total of 608 consecutive patients with PTC who were treated and clinically followed for PTC at Johns Hopkins Hospital between Jan. 1, 1990 and Dec. 31, 2015 were included in the present study. Data were analyzed from Jan. 30, 2019 to Jun. 18, 2023. All patients received total or near-total thyroidectomy. Clinicopathological data were collected from medical records. The pathological diagnoses of PTC were established according to World Health Organization criteria. Tumor stages were defined according to the AJCC Cancer Staging Manual (eight edition) staging system for thyroid cancer (44). Tumor recurrence in this study was defined as recurrent or persistent structural tumor existence diagnosed by imaging and confirmed by radioactive iodine scanning, biopsy or pathological examination. All tumor recurrence occurred within 13 years. Patient follow-up time was defined as the interval from initial thyroidectomy to the most recent clinical contact date or, in the case of patients with PTC recurrence, the date of discovery of disease recurrence. The study was approved by the institutional review board of Johns Hopkins University School of Medicine, and informed consent, when appropriate, was obtained from patients. This study followed the Strengthening the Reporting of Observational Studies in Epidemiology (STROBE) reporting guideline for cohort studies.

Mutational analyses. Genomic DNA was isolated from primary PTC using the standard phenol-chloroform extraction and ethanol precipitation procedures. Exon 15 of the BRAF gene and the core region of the TERT promoter were amplified using polymerase chain reaction testing for BigDye reaction followed by Sanger sequencing (22, 41). The SNP and TERT promoter mutations were sequenced in the same polymerase chain reaction test.

Cell lines and cell culture. Human PTC-derived cell line TPC1 (obtained from Dr. Alan P. Dackiw; Johns Hopkins University) was used to test activities of introduced TERT promoter in the pGL3 luciferase reporter constructs under various genetic conditions. Cells were cultured in RPMI-1640 medium with 10% fetal bovine serum (Gibco, ThermoFisher Scientific) at 37° C. in a humidified environment with 5% CO₂.

Luciferase reporter activity assay for TERT promoter. The pGL3 luciferase reporter constructs containing allele C of rs2853669 in the wild-type, C228T or C250T TERT promoter were generated as described previously (45). When desired, allele T of rs2853669 was generated in the above plasmids using the QuikChange Lightning Site-Directed Mutagenesis Kit (Agilent Technologies) with primers (forward: 5′-GCCACGTGGGAA GCGCGGTCCTGG-3′ (SEQ ID NO:16); reverse: 5′-CCAGGACCGCGCTTCCCACGTGGC-3′ (SEQ ID NO:17)).

For the promoter activity assay, TPC1 cells were seeded in triplicate on a 24-well plate and transfected with 300 ng of pGL3 luciferase reporter plasmids containing the indicated TERT promoter variant together with 12 ng of thymidine kinase promoter Renilla luciferase plasmid (normalizing control) using the jetPRIME transfection reagent (Polyplus). At 24 hours after transfection, cells were lysed and luciferase activities were measured using the Dual-Luciferase Reporter Assay System (Promega). Three independent experiments were conducted, and each was performed in triplicate. Results were reported as relative luciferase activities by dividing firefly luciferase values with Renilla luciferase values.

Statistical analysis. Continuous data were summarized using medians with IQRs or means with SDs, and categorical data were summarized using frequencies and percentages. Categorical variables were compared using the² test or, in the case of small samples, the Fisher's exact test. The Wilcoxon-Mann-Whitney test was used for non-normally distributed continuous variables, and the independent t test was used for normally distributed continuous variables. Kaplan-Meier survival curves with log-rank tests were used to compare recurrence-free survival by genetic status. Cox regression analyses were used to assess the associations of genetic variants with disease recurrence. The proportional-hazards assumption was tested on the basis of Schoenfeld residuals. Potential confounding variables, including patient age, sex, multifocality, tumor size, extrathyroidal invasion, vascular invasion, and lymph node metastasis, were adjusted. The threshold for statistical significance was 2-tailed P<0.05. Analyses were performed using Stata/SE software, version 10.1 for Windows (StataCorp LLC), and GraphPad Prism software, version 6.0 for Windows (GraphPad Software, Inc).

### Results

Association of SNP rs2853669C>T alone with the aggressiveness of PTC. The clinicopathological characteristics of the 608 patients with PTC included in the present study are presented in Table 4. Overall, the median (IQR) age was 47 (37-57) years; 427 patients (70.2%) were women, and 181 (29.8%) were men. The prevalence of BRAF V600E was 31.7% (193 patients), and the prevalence of the TERT promoter mutation was 11.5% (70 patients). The prevalence of the TT genotype of rs2853669 was 44.9% (273 patients), and the prevalence of the TC and CC (hereinafter, TC/CC) genotype was 55.1% (335 patients). All patients were followed up for a median (IQR) of 75 (36-123) months, representing 4232.25 person-years. The structural recurrence of PTC was 18.1% (110 patients), with a recurrence rate of 25.99 cases per 1000 person-years (95% CI, 21.56-31.33 cases per 1000 person-years). In the overall analysis of the entire cohort, there was generally no substantial difference in PTC characteristics between the TT genotype and the TC/CC genotype of rs2853669, except for significantly larger tumor size and insignificantly higher prevalence of distant metastasis and structural tumor recurrence in the former.

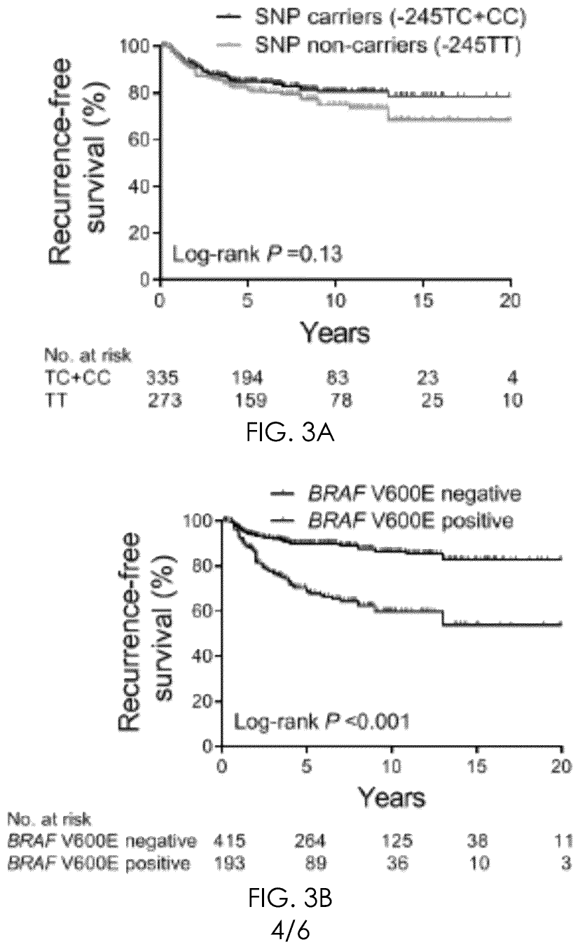

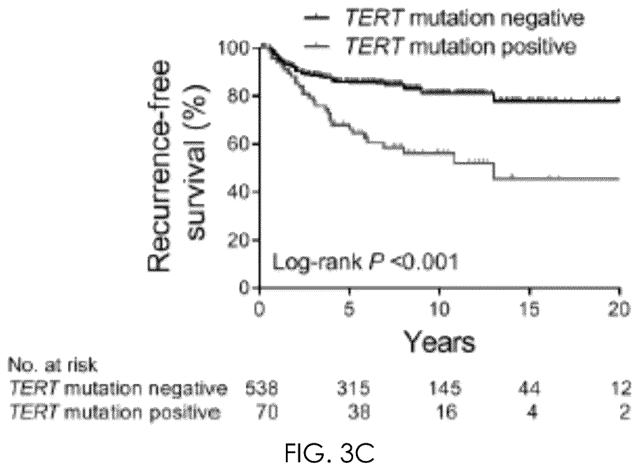

In Kaplan-Meier analyses of all cases, SNP rs2853669C>T was not associated with overall recurrence-free survival; patients with the TT genotype TT had a slightly higher rate of PTC recurrence compared with those with the TC/CC genotype (FIG. 3A). In contrast, BRAF V600E and TERT promoter mutation, were each associated with an accelerated decline in recurrence-free survival (FIGS. 3B and 3B). Cox proportional hazards analyses also revealed that SNP rs2853669C>T was not associated with PTC recurrence, with an adjusted hazard ratio (HR) of 0.85 (95% CI 0.57-1.27; P=0.43) for the TT versus TC/CC genotype (Table 5). BRAF V600E was associated with a significantly higher risk of PTC recurrence, with an adjusted HR of 2.11 (95% CI 1.37-3.26; P=0.001). The TERT promoter mutation had a significant unadjusted HR of 2.73 (1.77-4.20; P<0.001) and insignificant adjusted HR of 1.52 (95% CI 0.94-2.46; P=0.09).

Association of BRAF V600E and TERT promoter mutations with PTC recurrence risk by SNP rs2853669C>T genotype status. When the entire cohort of patients was divided into TT and TC/CC genotypes of rs2853669 to examine the risk of PTC recurrence, BRAF V600E had a significant adjusted HR of 3.15 (95% CI: 1.70-5.81, P<0.001) in the presence of the TT genotype and an insignificant adjusted HR of 1.54 (95% CI: 0.79-3.02, P=0.21) in the presence of the TC/CC genotype (Table 1). Similarly, the TERT promoter mutation had a significant adjusted HR of 2.12 (95% CI: 1.10-4.12, P=0.03) in the presence of the TT genotype, but an insignificant adjusted HR of 1.17 (95% CI: 0.56-2.41, P=0.68) in the presence of the TC/CC genotype (Table 1). These results suggested that although rs2853669 alone had no association with tumor recurrence, it modified the associations of the mutations: the TT genotype robustly cooperated with BRAF V600E and TERT promoter mutations to increase the risk of PTC recurrence, while the TC/CC genotype decreased or even eliminated the recurrence risk associated with the mutations.

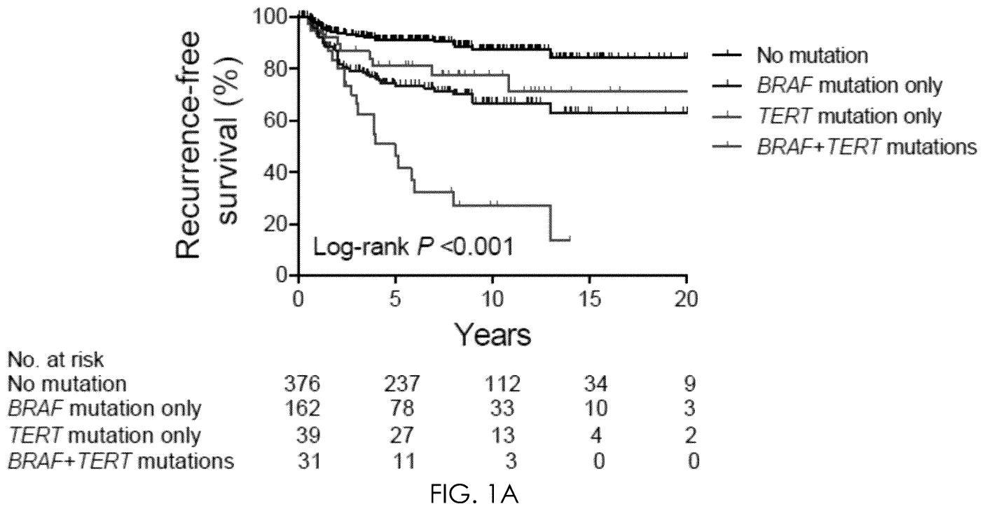

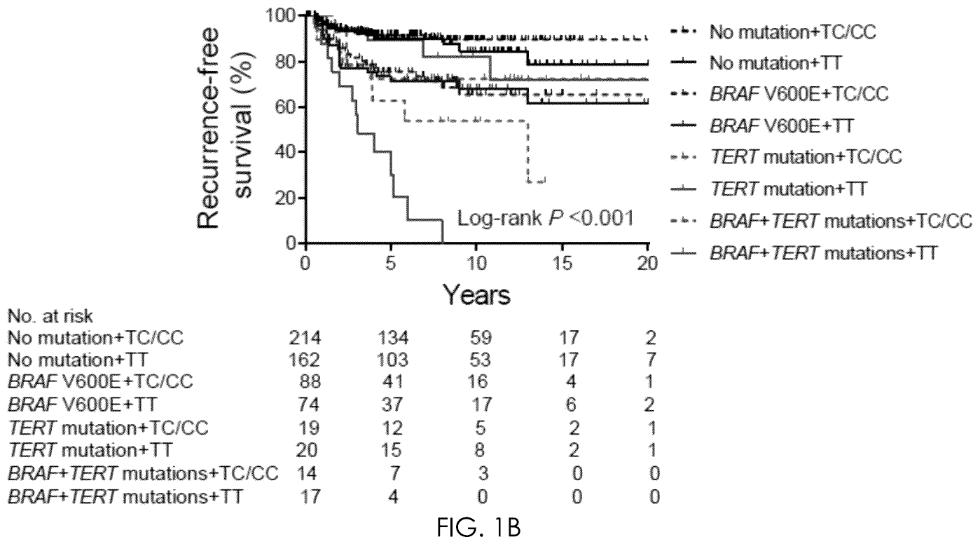

Consistent with previous findings (22), the present study found a robust synergism between BRAF V600E and TERT promoter mutations in increasing the risk of PTC recurrence (FIG. 1A and Table 6). Specifically, in the analysis of the entire patient cohort, recurrence rates were 64.5% (20 of 31 patients) in those harboring both variants versus 10.1% (38 of 376 patients) in those not harboring either variant, with an adjusted HR of 3.67 (95% CI, 1.75-7.70; P=0.001). Correspondingly, the recurrence-free survival curves revealed a moderate decline with BRAF V600E or TERT promoter mutation alone but a sharp decline with the genetic duet of the two coexisting mutations (FIG. 1A).

When dissecting BRAF V600E from the TERT promoter mutations and examining the association of SNP rs2853669C>T with tumor recurrence in patients with each mutation alone (without overlapping of the two mutations), those with the TT vs TC/CC genotype had no significant difference (Table 2). In contrast, among patients with the genetic duet of BRAF V600E and TERT promoter mutations, the TT genotype of rs2853669 was associated with significantly higher PTC recurrence compared with the TC/CC genotype, with an adjusted HR of 14.26 (95% CI: 2.86-71.25; P=0.001). These results suggested that the TT genotype of rs2853669 required the presence of both BRAF V600E and TERT promoter mutation to be implicated in the most aggressive PTC.

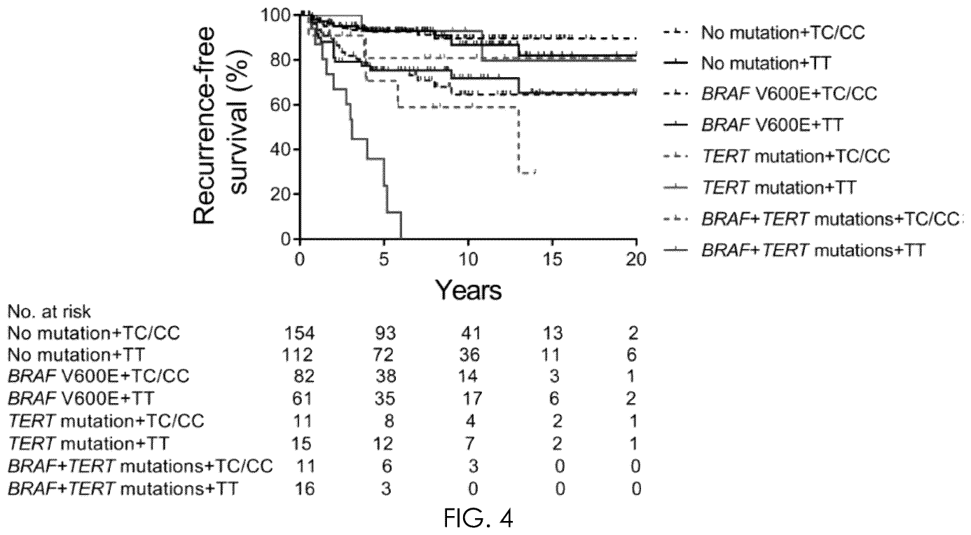

We further investigated the differentiating role of SNP rs2853669C>T in the prognostic precision of BRAF V600E and TERT promoter mutations by dividing patients into 8 genotype groups according to the genetic status of BRAF, TERT, and SNP rs2853669C>T (Table 3 and FIG. 1B). The risk of recurrence with the TT genotype was highest when coexisting with the genetic duet of BRAF V600E and TERT promoter mutations. The genetic trio of coexisting BRAF V600E, TERT promoter mutation and TT genotype of rs2853669 was associated with a recurrence rate of 76.5% (13 of 17 patients) versus 8.4% (18 of 214 patients) in those with the TC/C genotype who were not harboring either mutation, with an unadjusted HR of 13.48 (95% CI: 6.44-28.21; P<0.001); this HR remained significant at 6.96 (95% CI: 2.39-20.27; P<0.001) after multivariable adjustment (Table 3). In the presence of the TC/CC genotype of rs2853669, the genetic duet of BRAF V600E and TERT promoter mutation had a substantially lower unadjusted HR of 5.65 (95% CI: 2.36-13.54), which became insignificant at 1.19 (95% CI: 0.29-4.86) after multivariable adjustment. However, it should be noted that such adjustments are not entirely valid (46). From a biological perspective, if the clinicopathological characteristics (ie, tumor behaviors) are biologically promoted by the oncogenic mutations, to adjust them may artificially nullify the consequences of the mutations, resulting in a misleading underestimation of their biologically induced clinical impacts. Nevertheless, even after adjustment, the trio of coexisting BRAF V600E, TERT promoter mutation and TT genotype of rs2853669 still had a significant HR of 6.96 (2.39-20.27), suggesting a strong tumor-promoting function of the TT genotype compared with the TC/CC genotype when cooperating with BRAF V600E and TERT promoter mutations. Similar results were observed in conventional-variant PTC (Table 7 and FIG. 4).

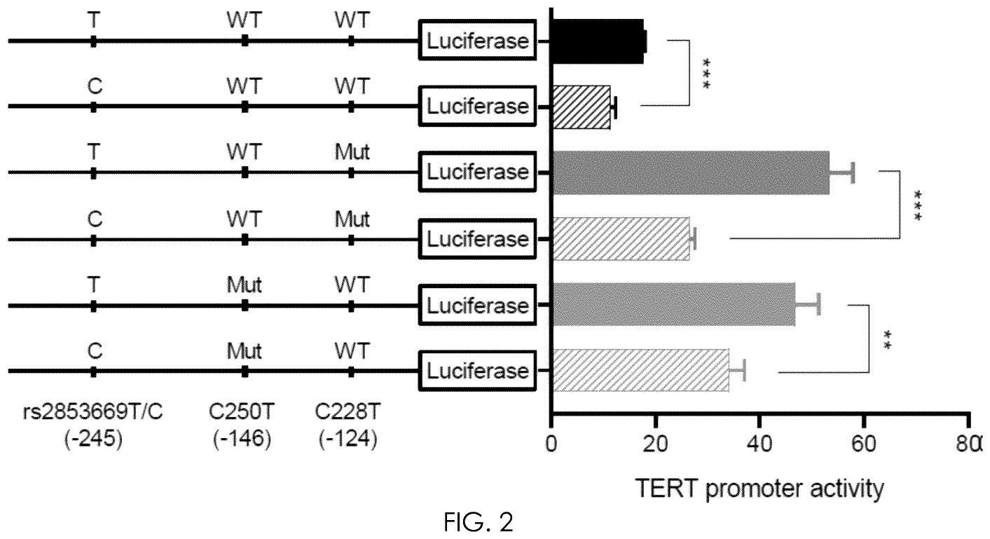

Modulation of TERT promoter activities by SNP rs2853669T>C. We used in vitro luciferase reporter assays to examine the relationship between SNP rs2853669C>T and TERT promoter activities with different genetic variants of the TERT promoter. The TERT promoter mutation increased the promoter activities to 2 to 3 times those of the wild-type TERT promoter (FIG. 2). Allele T of rs2853669 was associated with robustly higher TERT promoter activities compared with allele C; these higher activities were particularly prominent in the mutant TERT promoter. These results were consistent with and explained the observations of the differentiating roles of rs2853669 in the prognostic precision of BRAF V600E and TERT promoter mutations in estimating the risk of PTC recurrence.

### Discussion

This study demonstrated that the BRAF V600E and TERT promoter mutation-centered molecular prognostic strategy for estimating PTC outcomes could be refined to even higher precision by including SNP rs2853669T>C. Specifically, SNP rs2853669C>T could robustly differentiate the recurrence risk of PTC associated with BRAF V600E and TERT promoter mutations. In general, the TT genotype of rs2853669 synergized with the mutations, while the TC/CC genotype decreased and even eliminated the consequences of the mutations for PTC aggressiveness. A large meta-analysis also revealed that the TT genotype of rs2853669 in coexistence with the TERT promoter mutation was associated with poor clinical outcomes of some human cancers (40). SNP rs2853669C>T was found to particularly modify the prognostic precision of TERT promoter mutations for estimating outcomes in bladder cancer (41), glioblastoma (47, 48), clear cell renal cell carcinoma (49), and melanoma (50). Unlike the present study, these previous studies only examined the relationship of the SNP with the TERT promoter mutation, not the genetic duet of BRAF and TERT mutations. In fact, without the BRAF mutation, the SNP had a limited role in the prognostic value of the TERT promoter mutation.

The most notable finding in the present study was the association of SNP rs2853669C>T with the significantly greater prognostic precision of the genetic duet of BRAF V600E and TERT promoter mutations; this prognostic refining by SNP rs2853669C>T was more robust for the genetic duet of the two mutations than for the individual mutation alone. Specifically, the genetic duet of BRAF V600E and TERT promoter mutation was robustly associated with PTC recurrence in the presence of the TT genotype of rs2853669; patients harboring the trio of BRAF V600E, TERT promoter mutation, and TT genotype of rs2853669 had the worst clinical outcomes, while patients with the TC/CC genotype and neither mutation had the best prognosis. These data had important new prognostic implications beyond the current knowledge and prognostic use of BRAF V600E and TERT promoter mutations in PTC. The genetic duet of BRAF V600E and TERT promoter mutation and its association with tumor aggressiveness have been also reported in other cancers (51). The genetic trio of BRAF V600E, TERT promoter mutation, and TT genotype of rs2853669 likely also has prognostic value in other cancers (52).

The molecular mechanism underlying the interaction of SNP rs2853669C>T with BRAF V600E and TERT promoter mutations in affecting the aggressiveness of PTC remains to be elucidated but is likely associated with the regulatory machinery of the TERT promoter. SNP rs2853669C>T is in the ETS2 binding site; like the TERT mutation sites, the ETS2 binding site is located within the proximal core promoter region of the TERT gene. The present study found that allele T of rs2853669 was associated with robustly increased TERT promoter activities compared with allele C; allele T was required to sustain the full activities of the TERT promoter, particularly the mutant TERT promoter. Previous studies found that the ETS2 binding site in the TERT promoter was disrupted by allele C of rs2853669, resulting in the failure of ETS2 to bind the TERT promoter and hence the silencing of TERT expression (42, 43). A prominent mechanism for the activation of the mutant TERT promoter by BRAF V600E is the regulation of mutant TERT by BRAF V600E/MAPK/FOS through the GABP complex to act at the mutation site in the TERT promoter to upregulate the TERT gene (45). It is then compelling to speculate that the ETS2-linked regulatory machinery on the TERT promoter may cross talk with the regulatory system of BRAF V600E-MAPK-FOS-GABPB to cooperatively and oncogenically upregulate the mutant TERT promoter. This crosstalk could explain the association of the trio of BRAF V600E, TERT promoter mutation and TT genotype of rs2853669 with the worst clinical outcomes of PTC.

This study has limitations. We were unable to analyze the role of SNP rs2853669C>T in PTC-associated mortality due to its low incidence rate in the current study. Further studies involving larger cohorts are needed to address this issue. Moreover, this is a single-center study without replication, and the findings need to be validated in other cohorts.

In conclusion, this study found that SNP rs2853669T>C, through modulating the TERT promoter activities, substantially refined the prognostic precision of BRAF V600E and TERT promoter mutations, particularly that of the genetic duet, for estimating the risk of PTC recurrence. The trio of BRAF V600E, TERT promoter mutation and TT genotype of rs2853669 was associated with the highest recurrence of PTC while the lack of both mutations in the presence of the TC/CC genotype of rs2853669 was associated with the lowest recurrence. Combined use of these genetic variants of BRAF and TERT genes may be a simple but precise genetic-based risk prognostication strategy for PTC.

### REFERENCES

1. Mao Y, Xing M. Recent incidences and differential trends of thyroid cancer in the USA. Endocr Relat Cancer 2016; 23(4):313-22.
2. Lim H, Devesa S S, Sosa J A, et al. Trends in Thyroid Cancer Incidence and Mortality in the United States, 1974-2013. JAMA 2017; 317(13):1338-1348.
3. Haugen B R, Alexander E K, Bible K C, et al. 2015 American Thyroid Association Management Guidelines for Adult Patients with Thyroid Nodules and Differentiated Thyroid Cancer: The American Thyroid Association Guidelines Task Force on Thyroid Nodules and Differentiated Thyroid Cancer. Thyroid 2016; 26(1):1-133.
4. Schlumberger M, Leboulleux S. Current practice in patients with differentiated thyroid cancer. Nat Rev Endocrinol 2021; 17(3):176-188.
5. Xing M, Haugen B R, Schlumberger M. Progress in molecular-based management of differentiated thyroid cancer. Lancet 2013; 381(9871):1058-69.
6. Filetti S, Durante C, Hartl D, et al. Thyroid cancer: ESMO Clinical Practice Guidelines for diagnosis, treatment and follow-updagger. Ann Oncol 2019; 30(12):1856-1883.
7. Xing M. Genetic-guided Risk Assessment and Management of Thyroid Cancer. Endocrinol Metab Clin North Am 2019; 48(1):109-124.
8. Singh Ospina N, Iniguez-Ariza N M, Castro M R. Thyroid nodules: diagnostic evaluation based on thyroid cancer risk assessment. BMJ 2020; 368:16670.
9. Xing M. BRAF mutation in thyroid cancer. Endocr Relat Cancer 2005; 12(2):245-62.
10. Liu R, Xing M. TERT promoter mutations in thyroid cancer. Endocr Relat Cancer 2016; 23(3):R143-55.
11. Chung J H. BRAF and TERT promoter mutations: clinical application in thyroid cancer. Endocr J 2020; 67(6):577-584.
12. Bell R J, Rube H T, Kreig A, et al. The transcription factor GABP selectively binds and activates the mutant TERT promoter in cancer. Science 2015; 348(6238):1036-9.
13. Borah S, Xi L, Zaug A J, et al. TERT promoter mutations and telomerase reactivation in urothelial cancer. Science 2015; 347(6225):1006-10.
14. Xing M, Westra W H, Tufano R P, et al. BRAF mutation predicts a poorer clinical prognosis for papillary thyroid cancer. J Clin Endocrinol Metab 2005; 90(12):6373-9.
15. Xing M, Clark D, Guan H, et al. BRAF mutation testing of thyroid fine-needle aspiration biopsy specimens for preoperative risk stratification in papillary thyroid cancer. J Clin Oncol 2009; 27(18):2977-82.
16. Landa I, Ganly I, Chan T A, et al. Frequent somatic TERT promoter mutations in thyroid cancer: higher prevalence in advanced forms of the disease. J Clin Endocrinol Metab 2013; 98(9):E1562-6.
17. Liu X, Bishop J, Shan Y, et al. Highly prevalent TERT promoter mutations in aggressive thyroid cancers. Endocr Relat Cancer 2013; 20(4):603-10.
18. Xing M, Alzahrani A S, Carson K A, et al. Association between BRAF V600E mutation and mortality in patients with papillary thyroid cancer. JAMA 2013; 309(14):1493-501.
19. Liu R, Xing M. Diagnostic and prognostic TERT promoter mutations in thyroid fine-needle aspiration biopsy. Endocr Relat Cancer 2014; 21(5):825-30.
20. Liu X, Qu S, Liu R, et al. TERT promoter mutations and their association with BRAF V600E mutation and aggressive clinicopathological characteristics of thyroid cancer. J Clin Endocrinol Metab 2014; 99(6):E1130-6.
21. Melo M, da Rocha A G, Vinagre J, et al. TERT promoter mutations are a major indicator of poor outcome in differentiated thyroid carcinomas. J Clin Endocrinol Metab 2014; 99(5):E754-65.
22. Xing M, Liu R, Liu X, et al. BRAF V600E and TERT promoter mutations cooperatively identify the most aggressive papillary thyroid cancer with highest recurrence. J Clin Oncol 2014; 32(25):2718-26.
23. Xing M, Alzahrani A S, Carson K A, et al. Association between BRAF V600E mutation and recurrence of papillary thyroid cancer. J Clin Oncol 2015; 33(1):42-50.
24. Jin L, Chen E, Dong S, et al. BRAF and TERT promoter mutations in the aggressiveness of papillary thyroid carcinoma: a study of 653 patients. Oncotarget 2016; 7(14):18346-55.
25. Kim T H, Kim Y E, Ahn S, et al. TERT promoter mutations and long-term survival in patients with thyroid cancer. Endocr Relat Cancer 2016; 23(10):813-23.
26. Lee S E, Hwang T S, Choi Y L, et al. Prognostic Significance of TERT Promoter Mutations in Papillary Thyroid Carcinomas in a BRAF(V600E) Mutation-Prevalent Population. Thyroid 2016; 26(7):901-10.
27. Song Y S, Lim J A, Choi H, et al. Prognostic effects of TERT promoter mutations are enhanced by coexistence with BRAF or RAS mutations and strengthen the risk prediction by the ATA or TNM staging system in differentiated thyroid cancer patients. Cancer 2016; 122(9):1370-9.
28. Liu R, Bishop J, Zhu G, et al. Mortality Risk Stratification by Combining BRAF V600E and TERT Promoter Mutations in Papillary Thyroid Cancer: Genetic Duet of BRAF and TERT Promoter Mutations in Thyroid Cancer Mortality. JAMA Oncol 2017; 3(2):202-208.
29. Shen X, Liu R, Xing M. A six-genotype genetic prognostic model for papillary thyroid cancer. Endocr Relat Cancer 2017; 24(1):41-52.
30. Yang X, Li J, Li X, et al. TERT Promoter Mutation Predicts Radioiodine-Refractory Character in Distant Metastatic Differentiated Thyroid Cancer. J Nucl Med 2017; 58(2):258-265.
31. Rusinek D, Pfeifer A, Krajewska J, et al. Coexistence of TERT Promoter Mutations and the BRAF V600E Alteration and Its Impact on Histopathological Features of Papillary Thyroid Carcinoma in a Selected Series of Polish Patients. Int J Mol Sci 2018; 19(9).
32. Giorgenon T M V, Carrijo F T, Arruda M A, et al. Preoperative detection of TERT promoter and BRAFV600E mutations in papillary thyroid carcinoma in high-risk thyroid nodules. Arch Endocrinol Metab 2019; 63(2):107-112.
33. Huang M, Yan C, Xiao J, et al. Relevance and clinicopathologic relationship of BRAF V600E, TERT and NRAS mutations for papillary thyroid carcinoma patients in Northwest China. Diagn Pathol 2019; 14(1):74.
34. Liu J, Liu R, Shen X, et al. The Genetic Duet of BRAF V600E and TERT Promoter Mutations Robustly Predicts Loss of Radioiodine Avidity in Recurrent Papillary Thyroid Cancer. J Nucl Med 2020; 61(2):177-182.
35. Cao J, Zhu X, Sun Y, et al. The genetic duet of BRAF V600E and TERT promoter mutations predicts the poor curative effect of radioiodine therapy in papillary thyroid cancer. Eur J Nucl Med Mol Imaging 2022; 49(10):3470-3481.
36. Ngeow J, Eng C. TERT and BRAF in thyroid cancer: teaming up for trouble. J Clin Oncol 2014; 32(25):2683-4.
37. Moon S, Song Y S, Kim Y A, et al. Effects of Coexistent BRAF(V600E) and TERT Promoter Mutations on Poor Clinical Outcomes in Papillary Thyroid Cancer: A Meta-Analysis. Thyroid 2017; 27(5):651-660.
38. Vuong H G, Altibi A M A, Duong U N P, et al. Prognostic implication of BRAF and TERT promoter mutation combination in papillary thyroid carcinoma-A meta-analysis. Clin Endocrinol (Oxf) 2017; 87(5):411-417.
39. Ulisse S, Baldini E, Lauro A, et al. Papillary Thyroid Cancer Prognosis: An Evolving Field. Cancers (Basel) 2021; 13(21).
40. Shen N, Lu Y, Wang X, et al. Association between rs2853669 in TERT gene and the risk and prognosis of human cancer: a systematic review and meta-analysis. Oncotarget 2017; 8(31):50864-50872.
41. Rachakonda P S, Hosen I, de Verdier P J, et al. TERT promoter mutations in bladder cancer affect patient survival and disease recurrence through modification by a common polymorphism. Proc Natl Acad Sci USA 2013; 110(43):17426-31.
42. Hsu C P, Hsu N Y, Lee L W, et al. Ets2 binding site single nucleotide polymorphism at the hTERT gene promoter-effect on telomerase expression and telomere length maintenance in non-small cell lung cancer. Eur J Cancer 2006; 42(10):1466-74.
43. Xu D, Dwyer J, Li H, et al. Ets2 maintains hTERT gene expression and breast cancer cell proliferation by interacting with c-Myc. J Biol Chem 2008; 283(35):23567-80.
44. Perrier N D, Brierley J D, Tuttle R M. Differentiated and anaplastic thyroid carcinoma: Major changes in the American Joint Committee on Cancer eighth edition cancer staging manual. CA Cancer J Clin 2018; 68(1):55-63.
45. Liu R, Zhang T, Zhu G, et al. Regulation of mutant TERT by BRAF V600E/MAP kinase pathway through FOS/GABP in human cancer. Nat Commun 2018; 9(1):579.
46. Xing M. BRAF V600E mutation and papillary thyroid cancer. JAMA 2013; 310(5):535.
47. Spiegl-Kreinecker S, Lotsch D, Ghanim B, et al. Prognostic quality of activating TERT promoter mutations in glioblastoma: interaction with the rs2853669 polymorphism and patient age at diagnosis. Neuro Oncol 2015; 17(9):1231-40.
48. Batista R, Cruvinel-Carloni A, Vinagre J, et al. The prognostic impact of TERT promoter mutations in glioblastomas is modified by the rs2853669 single nucleotide polymorphism. Int J Cancer 2016; 139(2):414-23.
49. Hosen I, Rachakonda P S, Heidenreich B, et al. TERT promoter mutations in clear cell renal cell carcinoma. Int J Cancer 2015; 136(10):2448-52.
50. Chang G A, Robinson E, Wiggins J M, et al. Associations between TERT Promoter Mutations and Survival in Superficial Spreading and Nodular Melanomas in a Large Prospective Patient Cohort. J Invest Dermatol 2022; 142(10):2733-2743 e9.
51. Manrique-Silva E, Rachakonda S, Millan-Esteban D, et al. Clinical, environmental and histological distribution of BRAF, NRAS and TERT promoter mutations among patients with cutaneous melanoma: a retrospective study of 563 patients. Br J Dermatol 2021; 184(3):504-513.
52. Nagore E, Heidenreich B, Rachakonda S, et al. TERT promoter mutations in melanoma survival. Int J Cancer 2016; 139(1):75-84.

TABLE 1
Association of SNP rs2853669C > T genotype with the
prognostic precision of BRAF V600E and TERT promoter mutations

| | Recurrence rate | | 1000 person-year recurrence | | HR (95% CI) | |
|---|---|---|---|---|---|---|
| Genotypes | n/N (%) | P | Rate | 95% CI | Unadjusted | Adjusted* |
| TT of rs2853669 | | | | | | |
| Without BRAF V600E | 24/182 (13.2) | — | 16.78 | 11.25-25.03 | Ref. | Ref. |
| With BRAF V600E | 34/91 (37.4) | <0.001 | 63.19 | 45.15-88.43 | 3.41 (2.02-5.77) | 3.15 (1.70-5.81) |
| TC/CC of rs2853669 | | | | | | |
| Without BRAF V600E | 23/233 (9.9) | — | 13.79 | 9.16-20.75 | Ref. | Ref. |
| With BRAF V600E | 29/102 (28.4) | <0.001 | 48.65 | 33.81-70.01 | 3.21 (1.85-5.55) | 1.54 (0.79-3.02) |
| TT of rs2853669 | | | | | | |
| Without TERT mutation | 41/236 (17.4) | — | 23.65 | 17.41-32.12 | Ref. | Ref. |
| With TERT mutation | 17/37 (45.9) | <0.001 | 72.42 | 45.02-116.49 | 2.87 (1.63-5.05) | 2.12 (1.10-4.12) |
| TC/CC of rs2853669 | | | | | | |
| Without TERT mutation | 40/302 (13.2) | — | 19.69 | 14.44-26.84 | Ref. | Ref. |
| With TERT mutation | 12/33 (36.4) | <0.001 | 51.69 | 29.35-91.01 | 2.62 (1.38-5.00) | 1.17 (0.56-2.41) |

*Adjustment was made for patient age at diagnosis, sex, tumor multifocality, tumor size, extrathyroidal invasion, vascular invasion, and lymph node metastasis.

TABLE 2
Differentiating role of SNP rs2853669C > T genotype in the prognostic
precision of the genetic duet of BRAF V600E and TERT promoter mutation

| Mutation status | Recurrence rate | | 1000 person-year recurrence | | HR (95% CI) | |
|---|---|---|---|---|---|---|
| SNP status | n/N (%) | P | Rate | 95% (CI) | Unadjusted | Adjusted* |
| No mutation | | | | | | |
| TC/CC | 18/214 (8.4) | — | 11.80 | 7.43-18.72 | Ref. | Ref. |
| TT | 20/162 (12.3) | 0.21 | 16.00 | 10.32-24.80 | 1.42 (0.75-2.68) | 1.01 (0.46-2.22) |
| BRAF mutation only | | | | | | |
| TC/CC | 22/88 (25.0) | — | 43.49 | 28.63-66.04 | Ref. | Ref. |
| TT | 21/74 (28.4) | 0.63 | 43.40 | 28.30-66.57 | 1.07 (0.59-1.95) | 1.09 (0.58-2.06) |
| TERT mutation only | | | | | | |
| TC/CC | 5/19 (26.3) | — | 35.21 | 14.66-84.60 | Ref. | Ref. |
| TT | 4/20 (20.0) | 0.64 | 22.16 | 8.32-59.05 | 0.65 (0.17-2.44) | 0.0009 (1.23e−06-0.60) |
| BRAF + TERT mutation | | | | | | |
| TC/CC | 7/14 (50.0) | — | 77.63 | 37.01-162.85 | Ref. | Ref. |
| TT | 13/17 (76.5) | 0.13 | 239.63 | 139.14-412.69 | 3.30 (1.22-8.92) | 14.26 (2.86-71.25) |

*Adjustment was made for patient age at diagnosis, sex, multifocality, tumor size, extrathyroidal invasion, vascular invasion, and lymph node metastasis.

TABLE 3
Associations of BRAF V600E, TERT promoter mutation, and SNP rs2853669C > T genotype with the recurrence of PTC.

| | Recurrence rate | | 1000 person-year recurrence | | HR (95% CI) | |
|---|---|---|---|---|---|---|
| Mutation/SNP status | n/N (%) | P | Rate | 95% (CI) | Unadjusted | Adjusted* |
| No mutation + TC/CC | 18/214 (8.4) | — | 11.80 | 7.43-18.72 | Ref. | Ref. |
| No mutation + TT | 20/162 (12.3) | 0.21 | 16.00 | 10.32-24.80 | 1.42 (0.75-2.68) | 1.01 (0.46-2.22) |
| BRAF V600E + TC/CC | 22/88 (25.0) | <0.001 | 43.49 | 28.63-66.04 | 3.35 (1.80-6.25) | 2.26 (1.09-4.72) |
| BRAF V600E + TT | 21/74 (28.4) | <0.001 | 43.40 | 28.30-66.57 | 3.56 (1.90-6.69) | 2.27 (1.08-4.76) |
| TERT mutation + TC/CC | 5/19 (26.3) | 0.01 | 35.21 | 14.66-84.60 | 3.12 (1.16-8.40) | 3.78 (1.05-13.61) |
| TERT mutation + TT | 4/20 (20.0) | 0.10 | 22.16 | 8.32-59.05 | 2.09 (0.71-6.19) | 0.79 (0.17-3.64) |
| BRAF + TERT mutations + TC/CC | 7/14 (50.0) | <0.001 | 77.63 | 37.01-162.85 | 5.65 (2.36-13.54) | 1.19 (0.29-4.86) |
| BRAF + TERT mutations + TT | 13/17 (76.5) | <0.001 | 239.63 | 139.14-412.69 | 13.48 (6.44-28.21) | 6.96 (2.39-20.27) |

*Adjustment was made for patient age at diagnosis, sex, multifocality, tumor size, extrathyroidal invasion, vascular invasion, and lymph node metastasis.

TABLE 4
Relationship between SNP rs2853669C > T
and clinicopathological outcomes of the PTC cases

| | | TERT SNP rs2853669T > C | | |
|---|---|---|---|---|
| | Overall | TT | TC/CC | P |
| No. | 608 | 273 (44.9) | 335 (55.1) | |
| Age | 47 (37-57) | 46 (36-58) | 47 (38-57) | 0.31 |
| Sex, male | 181 (29.8) | 76 (27.8) | 105 (31.3) | 0.35 |
| Tumor size, cm | 1.5 (0.9-2.5) | 1.5 (0.9-2.6) | 1.4 (0.8-2.3) | 0.04 |
| Multifocality | 226 (37.2) | 92 (33.7) | 134 (40.0) | 0.11 |
| Extrathyroidal Invasion | 106 (17.4) | 50 (18.3) | 56 (16.7) | 0.61 |
| Vascular invasion | 92 (15.1) | 39 (14.3) | 53 (15.8) | 0.60 |
| Lymph node metastasis | 191 (31.4) | 87 (31.9) | 104 (31.0) | 0.83 |
| Distant metastasis | 38 (6.3) | 22 (8.1) | 16 (4.8) | 0.10 |
| Stage III + IV | 66 (10.9) | 32 (11.7) | 34 (10.1) | 0.54 |
| Structural tumor recurrence | 110 (18.1) | 58 (21.2) | 52 (15.5) | 0.07 |
| Follow-up time, months | 75 (36-123) | 72 (36-129) | 76 (32-119) | 0.53 |
| Total I-131 dose, mCi | 75 (0-100) | 75 (0-100) | 75 (0-100) | 0.83 |

TABLE 5
Relationship of BRAF V600E, TERT promoter mutation, and SNP rs2853669C > T with PTC recurrence

| | Recurrence rate | | 1000 person-year recurrence | | HR (95% CI) | | |
|---|---|---|---|---|---|---|---|
| Genetic status | n/N (%) | P | Rate | 95% (CI) | Unadjusted | Adjustment 1 | Adjustment 2 |
| Genotype of | | | | | | | |
| rs2853669 | | | | | | | |
| TT | 58/273 (21.2) | — | 29.47 | 22.78-38.11 | Ref. | Ref. | Ref. |
| TC/CC | 52/335 (15.5) | 0.07 | 22.97 | 17.50-30.14 | 0.75 (0.52-1.09) | 0.73 (0.50-1.07) | 0.85 (0.57-1.27) |
| BRAF V600E | | | | | | | |
| Negative | 47/415 (11.3) | — | 15.17 | 11.40-20.19 | Ref. | Ref. | Ref. |
| Positive | 63/193 (32.6) | <0.001 | 55.55 | 43.39-71.11 | 3.33 (2.28-4.86) | 3.32 (2.27-4.84) | 2.11 (1.37-3.26) |
| TERT mutation | | | | | | | |
| Negative | 81/538 (15.1) | — | 21.51 | 17.30-26.75 | Ref. | Ref. | Ref. |
| Positive | 29/70 (41.4) | <0.001 | 62.11 | 43.16-89.38 | 2.79 (1.83-4.27) | 2.73 (1.77-4.20) | 1.52 (0.94-2.46) |

Adjustment 1 was made for patient age at diagnosis and sex.
Adjustment 2 was made for patient age at diagnosis, sex, multifocality, tumor size, extrathyroidal invasion, vascular invasion, and lymph node metastasis.

TABLE 6
Hazard ratios of BRAF V600E, TERT promoter mutations, or their coexistence for PTC recurrence.

| | Recurrence rate | | 1000 person-year recurrence | | HR (95% CI) | |
|---|---|---|---|---|---|---|
| Mutation status | n/N (%) | P | Rate | 95% (CI) | Unadjusted | Adjusted* |
| No mutation | 38/376 (10.1) | — | 13.69 | 9.96-18.81 | Ref. | Ref. |
| BRAF mutation only | 43/162 (26.5) | <0.001 | 43.44 | 32.22-58.58 | 2.93 (1.90-4.54) | 2.14 (1.30-3.51) |
| TERT mutation only | 9/39 (23.1) | 0.02 | 27.91 | 14.52-53.63 | 2.16 (1.04-4.47) | 1.51 (0.68-3.37) |
| BRAF + TERT mutation | 20/31 (64.5) | <0.001 | 138.49 | 89.35-214.66 | 8.10 (4.68-14.01) | 3.67 (1.75-7.70) |

*Adjustment was made for patient age at diagnosis, sex, multifocality, tumor size, extrathyroidal invasion, vascular invasion, and lymph node metastasis.

TABLE 7
Association of BRAF V600E, TERT promoter mutation, and SNP rs2853669C > T with the recurrence of conventional PTC.

| | Recurrence rate | | 1000 person-year recurrence | | HR (95% CI) | |
|---|---|---|---|---|---|---|
| Mutation/SNP status | n/N (%) | P | Rate | 95% (CI) | Unadjusted | Adjusted* |
| No mutation + TC/CC | 12/154 (7.8) | — | 11.10 | 6.30-19.55 | Ref. | Ref. |
| No mutation + TT | 11/112 (9.8) | 0.56 | 12.56 | 6.95-22.68 | 1.19 (0.52-2.69) | 1.12 (0.41-3.07) |
| BRAF V600E + TC/CC | 21/82 (25.6) | <0.001 | 44.58 | 29.07-68.37 | 3.61 (1.78-7.34) | 2.47 (1.10-5.57) |
| BRAF V600E + TT | 16/61 (26.2) | <0.001 | 36.06 | 22.09-58.85 | 3.31 (1.57-7.00) | 1.87 (0.80-4.38) |
| TERT mutation + TC/CC | 2/11 (18.2) | 0.24 | 19.59 | 4.90-78.34 | 2.05 (0.46-9.18) | 1.23 (0.17-8.78) |
| TERT mutation + TT | 2/15 (13.3) | 0.36 | 13.31 | 3.33-53.22 | 1.38 (0.31-6.21) | 0.44 (0.05-4.03) |
| BRAF + TERT mutations + TC/CC | 5/11 (45.5) | <0.001 | 66.30 | 27.60-159.28 | 5.25 (1.85-14.90) | 2.10 (0.35-12.48) |
| BRAF + TERT mutations + TT | 12/16 (75.0) | <0.001 | 259.46 | 147.35-456.87 | 17.08 (7.16-40.70) | 8.66 (2.49-30.15) |

*Adjustment was made for patient age at diagnosis, sex, multifocality, tumor size, extrathyroidal invasion, vascular invasion, and lymph node metastasis.

That which is claimed:

1. A method for treating a subject having aggressive thyroid cancer comprising the steps of:
a. performing an assay on a sample obtained from the subject to identify a
(i) a T1799A mutation in the v-raf murine sarcoma viral oncogene homolog B1 (BRAF) gene that results in a V600E amino acid change,
(ii) a genotype of TT at the single nucleotide polymorphism (SNP) rs2853669, and either
(iii) a 1 295 228 C>T (C228T) mutation, corresponding to −124 C>T from the translation start site in the promoter of the telomerase reverse transcriptase (TERT) gene, or
(iv) a 1 295 250 C>T (C250T) mutation, corresponding to −146 C>T from the translation start site in the promoter of TERT;
b. identifying the subject as having or likely to develop aggressive thyroid cancer when the V600E mutation, genotype of TT at rs2853669 and either the C228T or C250T mutations are identified; and
c. treating the subject with one or more treatment modalities appropriate for a subject having or likely to develop aggressive thyroid cancer.

2. The method of claim 1, wherein the assay of step (a) comprises sequencing of the BRAF gene that comprises the T1799A nucleotide site and sequencing of the TERT promoter region comprising −124, −146 and −245 from the translation start site in the promoter of TERT.

3. The method of claim 1, wherein the assay of step (a) comprises the steps of:
i. extracting DNA from the biological sample;
ii. contacting the DNA with a primer that specifically hybridizes to the BRAF gene and a primer that specifically hybridizes to the TERT gene;
iii. amplifying by polymerase chain reaction (PCR) a region of the BRAF gene that comprises the T1779A nucleotide site and a region of the TERT gene that comprises −124, −146 and −245 from the translation start site in the promoter of TERT; and
iv. sequencing the amplification product to identify the presence of the V600E mutation, genotype of TT at rs2853669, and either the C228T or C250T mutation.

4. The method of claim 1, wherein the treatment modality for aggressive thyroid cancer comprises thyroidectomy, hemithyroidectomy, radioactive iodine therapy, and combinations thereof.

5. The method of claim 1, wherein the treatment modality comprises administering to the subject a BRAF V600E inhibitor.

6. The method of claim 1, wherein the treatment modality comprises administering to the subject a MEK inhibitor.

7. The method of claim 1, wherein the treatment modality comprises administering to the subject a TERT inhibitor.

8. The method of claim 1, wherein the treatment modality comprises administering to the subject a FOS inhibitor.

9. The method of claim 1, wherein the treatment modality comprises administering to the subject both BRAF V600E/MEK inhibitors and TERT inhibitor.

10. The method of claim 1, wherein the aggressive thyroid cancer is papillary thyroid cancer (PTC) or anaplastic thyroid cancer.

11. A method for identifying a subject as having or likely to develop aggressive thyroid cancer comprising the steps of:
a. performing an assay on a sample obtained from the subject to identify a
(i) a T1799A mutation in the BRAF gene that results in a V600E amino acid change,
(ii) a genotype of TT at the SNP rs2853669, and either
(iii) a 1 295 228 C>T (C228T) mutation, corresponding to −124 C>T from the translation start site in the promoter of the TERT gene, or
(iv) a 1 295 250 C>T (C250T) mutation, corresponding to −146 C>T from the translation start site in the promoter of TERT; and
b. identifying the subject as having or likely to develop aggressive thyroid cancer when the V600E mutation, genotype of TT at rs2853669 and either the C228T or C250T mutations are identified.

12. The method of claim 11, wherein the assay of step (a) comprises sequencing of the BRAF gene that comprises the T1799A nucleotide site and sequencing of the TERT promoter region comprising −124, −146 and −245 from the translation start site in the promoter of TERT.

13. The method of claim 11, wherein the assay of step (a) comprises the steps of:
i. extracting DNA from the biological sample;
ii. contacting the DNA with a primer that specifically hybridizes to the BRAF gene and a primer that specifically hybridizes to the TERT gene;
iii. amplifying by polymerase chain reaction (PCR) a region of the BRAF gene that comprises the T1779A nucleotide site and a region of the TERT gene that comprises −124, −146 and −245 from the translation start site in the promoter of TERT; and
iv. sequencing the amplification product to identify the presence of the V600E mutation, genotype of TT at rs2853669, and either the C228T or C250T mutation.

14. The method of claim 11, further comprising the step of administering a treatment modality appropriate for a subject having or likely to develop aggressive thyroid cancer.

15. The method of claim 14, wherein the treatment modality for aggressive thyroid cancer comprises thyroidectomy, hemithyroidectomy, radioactive iodine therapy, and combinations thereof.

16. The method of claim 14, wherein the treatment modality comprises administering to the subject a BRAF V600E inhibitor.

17. The method of claim 14, wherein the treatment modality comprises administering to the subject a TERT inhibitor.

18. The method of claim 14, wherein the treatment modality comprises administering to the subject a FOS inhibitor.

19. The method of claim 11, wherein the aggressive thyroid cancer is papillary thyroid cancer (PTC) or anaplastic thyroid cancer.

20. The method of claim 14, wherein the treatment modality comprises administering to the subject a MEK inhibitor.

21. The method of claim 14, wherein the treatment modality comprises administering to the subject both BRAF V600E/MEK inhibitors and TERT inhibitor.

22. The method of any one of claims 1-21, wherein the biological sample is from a fine needle aspiration biopsy.

23. The method of claim 3 or 13, wherein the amplification of the TERT gene is accomplished using one or more primers shown in SEQ ID NOS:1-7.

24. The method of claim 3 or 13, wherein the amplification of the BRAF gene is accomplished using on or more primers shown in SEQ ID NOS:8-13.


SEQUENCE LISTING (17 sequences)

SEQ ID NO: 1 - DNA, 20 bp, synthetic construct
  source 1..20: mol_type = other DNA
agtggattcgcgggcacaga

SEQ ID NO: 2 - DNA, 18 bp, synthetic construct
  source 1..18: mol_type = other DNA
cagcgctgcctgaaactc

SEQ ID NO: 3 - DNA, 18 bp, synthetic construct
  source 1..18: mol_type = other DNA
agcacctcgcggtagtgg

SEQ ID NO: 4 - DNA, 23 bp, synthetic construct
  source 1..23: mol_type = other DNA
cacccgtcctgccccttcacctt

SEQ ID NO: 5 - DNA, 23 bp, synthetic construct
  source 1..23: mol_type = other DNA
ggcttcccacgtgcgcagcagga

SEQ ID NO: 6 - DNA, 21 bp, synthetic construct
  source 1..21: mol_type = other DNA
ccaagttcctgcactggctga

SEQ ID NO: 7 - DNA, 18 bp, synthetic construct
  source 1..18: mol_type = other DNA
ttcccgatgctgcctgac

SEQ ID NO: 8 - DNA, 23 bp, synthetic construct
  source 1..23: mol_type = other DNA
tcataatgcttgctctgatagga

SEQ ID NO: 9 - DNA, 22 bp, synthetic construct
  source 1..22: mol_type = other DNA
ggccaaaaatttaatcagtgga

SEQ ID NO: 10 - DNA, 48 bp, synthetic construct
  source 1..48: mol_type = other DNA
tgtaaaacgacggccagtctgttttcctttacttactacacctcagat

SEQ ID NO: 11 - DNA, 20 bp, synthetic construct
  source 1..20: mol_type = other DNA
caactgttcaaactgatggg

SEQ ID NO: 12 - DNA, 24 bp, synthetic construct
  source 1..24: mol_type = other DNA
aactcttcataatgcttgctctga

SEQ ID NO: 13 - DNA, 27 bp, synthetic construct
  source 1..27: mol_type = other DNA
cagacaactgttcaaactgatgggacc

SEQ ID NO: 14 - DNA, 27 bp, synthetic construct
  source 1..27: mol_type = other DNA
aataggtgattttggtctagctacagt

SEQ ID NO: 15 - DNA, 27 bp, synthetic construct
  source 1..27: mol_type = other DNA
aataggtgattttggtctagctacaga

SEQ ID NO: 16 - DNA, 24 bp, synthetic construct
  source 1..24: mol_type = other DNA
gccacgtgggaagcgcggtcctgg

SEQ ID NO: 17 - DNA, 24 bp, synthetic construct
  source 1..24: mol_type = other DNA
ccaggaccgcgcttcccacgtggc